(19) United States
(12) Patent Application Publication
(10) Pub. No.: US 2015/0067922 A1
(43) Pub. Date: Mar. 5, 2015

(54) GENE TARGETING AND GENETIC MODIFICATION OF PLANTS VIA RNA-GUIDED GENOME EDITING

(71) Applicant: The Penn State Research Foundation, University Park, PA (US)

(72) Inventors: Yinong Yang, State College, PA (US); Kabin Xie, State College, PA (US)

(73) Assignee: The Penn State Research Foundation, University Park, PA (US)

(21) Appl. No.: 14/291,605

(22) Filed: May 30, 2014

Related U.S. Application Data
(60) Provisional application No. 61/828,737, filed on May 30, 2013.

(51) Int. Cl. C12N 15/82

(52) U.S. Cl. CPC C12N 15/8289; C12N 15/8274; C12N 15/8273; C12N 15/8286; C12N 15/8271; C12N 15/8247; C12N 15/8245; C12N 15/8281; C12N 15/8282; C12N 15/8283; C12N 15/8261
USPC 800/298; 435/468; 435/469; 435/419; 435/320.1

(57) ABSTRACT

The present invention provides compositions and methods for specific gene targeting and precise editing of DNA sequences in plant genomes using the CRISPR (cluster regularly interspaced short palindromic repeats) associated nuclease. Non-transgenic, genetically modified crops can be produced using these compositions and methods.

### CROSS-REFERENCE TO RELATED APPLICATIONS

This application claims priority under 35 U.S.C. §119 to provisional application Ser. No. 61/828,737 filed May 30, 2013, herein incorporated by reference in its entirety.

### STATEMENT REGARDING FEDERALLY FUNDED RESEARCH

This invention was made with government support under Hatch Act Project No. PEN04256, awarded by the United States Department of Agriculture. The Government has certain rights in the invention.

### FIELD OF THE INVENTION

This invention relates to methods for plant gene targeting and genome editing in the field of molecular biology and genetic engineering. More specifically, the invention describes the use of CRISPR-associated nuclease to specifically and efficiently edit DNA sequences of the plant genome for genetic engineering.

### BACKGROUND OF THE INVENTION

Methodologies for specific gene targeting or precise genome editing are of great importance to functional characterization of plant genes and genetic improvement of agricultural crops. In contrast to microbial and mammalian systems in which gene targeting is an established tool, it is extremely inefficient and difficult to achieve successful gene targeting in plants, largely due to the low frequency of homologous recombination. Therefore, it is imperative to develop new technologies for more efficient and specific gene targeting and genome editing in plants.

In recent years, sequence-specific nucleases have been developed to increase the efficiency of gene targeting or genome editing in animal and plant systems. Among them, zinc finger nucleases (ZFNs) and transcription activator-like effector nucleases (TALENs) are the two most commonly used sequence-specific chimeric proteins. Once the ZFN or TALEN constructs are introduced into and expressed in cells, the programmable DNA binding domain can specifically bind to a corresponding sequence and guide the chimeric nuclease (e.g., the FokI nuclease) to make a specific DNA strand cleavage. A pair of ZFNs or TALENs can be introduced to generate double strand breaks (DSBs), which activate the DNA repair systems and significantly increase the frequency of both nonhomologous end joining (NHEJ) and homologous recombination (HR).

In general, single zinc-finger motif specifically recognizes 3 bp, and engineered zinc-finger with tandem repeats can recognize up to 9-36 bp. However, it is quite tedious and time-consuming to screen and identify a desirable ZFN. Despite its drawbacks, ZFN has been used in plants to introduce small mutations, gene deletion, or foreign DNA integration (gene replacement/knock-in) at the specific genomic site. In contrast with the zinc finger protein, TALEs are derived from the plant pathogenic bacteria Xanthomonas and contain 34 amino acid tandem repeats in which repeat-variable diresidues (RVDs) at positions 12 and 13 determine the DNA-binding specificity. As a result, TALENs with 16-24 tandem repeats can specifically recognize 16-24 by genomic sequences and the chimeric nuclease can generate DSBs at specific genomic sites. TALEN-mediated genome editing has already been demonstrated in many organisms including yeast, animals, and plants.

Most recently, a new gene targeting tool has been developed in microbial and mammalian systems based on the cluster regularly interspaced short palindromic repeats (CRISPR)-associated nuclease system. The CRISPR-associated nuclease is part of adaptive immunity in bacteria and archaea. The Cas9 endonuclease, a component of Streptococcus pyogenes type II CRISPR/Cas system, forms a complex with two short RNA molecules called CRISPR RNA (crRNA) and transactivating crRNA (transcrRNA), which guide the nuclease to cleave non-self DNA on both strands at a specific site. The crRNA-transcrRNA heteroduplex could be replaced by one chimeric RNA (so-called guide RNA (gRNA)), which can then be programmed to targeted specific sites. The minimal constrains to program gRNA-Cas9 is at least 15-base-pairing between engineered 5′-RNA and targeted DNA without mismatch, and an NGG motif (so-called protospacer adjacent motif or PAM) follows the base-pairing region in the targeted DNA sequence. Generally, 15-22 nt in the 5′-end of the gRNA region is used to direct Cas9 nuclease to generate DSBs at the specific site. The CRISPR/Cas system has been demonstrated for genome editing in human, mice, zebrafish, yeast and bacteria. Distinct from animal, yeast, or bacterial cells to which recombinant molecules (DNA, RNA or protein) could be directly transformed for Cas9-mediated genome editing, recombinant plasmid DNA is typically delivered into plant cells via the Agrobacterium-mediate transformation, biolistic bombardment, or protoplast transformation due to the presence of cell wall. Thus, specialized molecular tools and methods need to be created to facilitate the construction and delivery of plasmid DNAs as well as efficient expression of Cas9 and gRNAs for genome editing in plants. Furthermore, Cas9-gRNA recognizes target sequence based on the gRNA and DNA base pairing that may have a risk of off-targeting. Therefore it is also critical to determine the parameter for designing Cas9-gRNA constructs with minimal off-target risk for plant genome editing. Due to these significant differences between animals and plants, it is still unknown if the CRISPR-Cas system is functional in the plant system and if it can be exploited for specific gene targeting and genome editing in crop species.

Compositions and methods for making and using CRISPR-Cas systems are described in U.S. Pat. No. 8,697,359, entitled “CRISPR-CAS SYSTEMS AND METHODS FOR ALTERING EXPRESSION OF GENE PRODUCTS,” which is incorporated herein in its entirety.

Therefore, it is a primary object, feature, or advantage of the present invention to improve upon the state of the art.

It is a further objective, feature, or advantage of the present invention to provide compositions and methods for gene targeting and genome editing in plants.

It is a further objective, feature or advantage of the present invention to provide compositions and methods for targeting specific genes in plants for gene editing.

It is a further objective, feature or advantage of the present invention to provide plasmid vector constructs that allow for gene targeting and genome editing in plants.

It is a further objective, feature or advantage of the present invention to provide compositions and methods for making and using a CRISPR-Cas system for gene targeting and gene editing in plants.

It is a further objective, feature or advantage of the present invention to provide novel promoters for use in driving expression of a gene or gene product of interest in a plant.

It is a further objective, feature or advantage of the present invention to provide novel parameters to minimize off-targeting of CRISPR-Cas system in plants.

Additional objectives, features and advantages may become obvious based on the disclosure contained herein.

### SUMMARY OF THE INVENTION

This invention provides materials and methods for specific gene targeting and precise genome editing in plant and crop species. In one embodiment, the CRISPR/Cas9 system is adapted to use in plants. In one embodiment, a series of plant-specific RNA-guided Genome Editing vectors (pRGE plasmids) are provided for expression of the CRISPR/Cas9 system in plants. The plasmids may be optimized for transient expression of the CRISPR/Cas9 system in plant protoplasts, or for stable integration and expression in intact plants via the Agrobacterium-mediated transformation. In one aspect, the plasmid vector constructs include a nucleotide sequence comprising a DNA-dependent RNA polymerase III promoter, wherein said promoter operably linked to a gRNA molecule and a Pol III terminator sequence, wherein said gRNA molecule includes a DNA target sequence; and a nucleotide sequence comprising a DNA-dependent RNA polymerase II promoter operably linked to a nucleic acid sequence encoding a type II CRISPR-associated nuclease.

According to one aspect of the invention, the inventors have identified critical parameters necessary for use of the gene editing technology in plants. In one aspect, it is critical to use promoters to drive expression of the CRISPR/Cas9 system at high levels in plants. In a further aspect, the type of promoter is dictated by the type of plant being targeted. In embodiment, the promoter driving expression of the gRNA molecule is critically dictated by the type of plant being targeted, for example, gene editing in a monocot requires use of a monocot promoter driving gRNA expression, and gene editing in a dicot requires use of a dicot promoter driving gRNA expression. In an exemplary embodiment, the promoter is the novel rice UBI10 promoter (OsUBI10 promoter, SEQ ID NO:1).

In one exemplary embodiment, compositions and methods are provided for gene targeting and gene editing of monocot species of plant, including rice, a model plant and crop species. In other embodiments, compositions and methods are provided for gene targeting and gene editing of dicot plants, including for example soybean (Glycine max), potato (Solanum), and Arabidopsis thaliana.

The materials and methods are applicable to any plant species, including for example various dicot and monocot crops including, such as tomato, cotton, maize (Zea mays), wheat, Arabidopsis thaliana, Medicago truncatula, Solanum lycopersicum, Glycine max, Brachypodium distachyon, Oryza sativa, Sorghum bicolor, or Solanum tuberosum.

According to one embodiment, materials and methods are provided for transient expression of the CRISPR/Cas9 system in plant protoplasts. In a preferred embodiment, plasmid vector constructs are disclosed for transient expression of CRISPR/Cas9 system in plant protoplasts. In a more preferred embodiment, the vector for transient transformation of plants is pRGE3 (SEQ ID NO:2), pRGE6 (SEQ ID NO:4), pRGE31 (SEQ ID NO:6), or pRGE32 (SEQ ID NO:8). In another preferred embodiment, the vector may be optimized for use in a particular plant type or species. In a preferred embodiment, the vector is pStGE3 (SEQ ID NO:10).

According to one embodiment, a CRISPR/Cas system on the binary vectors can be stably integrated into the plant genome, for example via Agrobacterium-mediated transformation. Thereafter, the CRISPR/Cas transgene can be removed by genetic cross and segregation, leading to the production of non-transgenic, but genetically modified plants or crops. In a preferred embodiment, the vector is optimized for Agrobacterium-mediated transformation. In a more preferred embodiment, the vector for stable integration is pRGEB3 (SEQ ID NO:3), pRGEB6 (SEQ ID NO:5), pRGEB31 (SEQ ID NO:7), pRGEB32 (SEQ ID NO:9), or pStGEB3 (SEQ ID NO:11).

In one aspect, gene editing may be obtained using the present invention via deletion or insertion. In another aspect, a donor DNA fragment with positive (e.g., herbicide or antibiotic resistance) and/or negative (e.g., toxin genes) selection markers could be co-introduced with the CRISPR/Cas system into plant cells for targeted gene repair/correction and knock-in (gene insertion and replacement) via homologous recombination. In combination with different donor DNA fragments, the CRISPR/Cas system could be used to modify various agronomic traits for genetic improvement.

Since the specificity of the CRISPR/Cas system is based on nucleotide pairing rather than the protein-DNA interaction, this method is likely much simpler, more specific, and more effective than the existing ZFN and TALEN systems for genome editing in plants. This technology will facilitate a new generation of various plant and crop cultivars with improved agronomic traits such as herbicide resistance, disease resistance, abiotic stress tolerance, high yield, superior crop quality, etc. In addition, non-transgenic approaches can be designed with this genome editing method, which should significantly improve public acceptance of genetically engineered plants.

In another aspect, the invention provides novel nucleotide sequences for use in driving expression of a gene or gene product of interest. In a preferred embodiment, a novel rice promoter (UBI10, SEQ ID NO:1) is provided. The novel promoter may be used to drive expression of a gene or gene product of interest in a plant, including monocot and dicot plants. According to a preferred embodiment, the promoter may be used to drive expression of Cas9 for a CRISPR/Cas gene editing system.

In another aspect, the invention provides novel parameters for Cas9-gRNA targeting specificity. In a preferred embodiment, parameter for specific gRNA design is provided.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### DESCRIPTION OF THE DRAWINGS

FIG. 1 shows a schematic description of Cas9 guided genome editing. The secondary structure of gRNA mimics the crRNA-transcrRNA heteroduplex that binds to Cas9. The 5′-end of gRNA is shown paired with one strand of a targeted DNA. A PAM motif (N-G-G) is located at the DNA-gRNA pairing region in the complementary strand of targeted DNA. The DNA-gRNA base pairing should be at least 15 by long. The Cas9 nuclease would cleave both strands of DNA at conserved position which is 3 by to the PAM motif.

FIG. 2(A-C) shows a diagram of pRGE vectors for transient expression. A DNA-dependent RNA polymerase III (Pol III) promoter and Pol III terminator are used to control the transcription of engineered gRNA. Rice Pol III promoters (snoRNA U3 and U6 promoters) were isolated to make pRGE3 (B) and pRGE6 (C) vectors. Plant DNA-dependent RNA polymerase type II (Pol II) and Pol II terminator are used to control the expression of a chimeric Cas9 nuclease. hSpCas9 encodes a human codon optimized Cas9 nuclease which includes a nuclear localization signal (NLS) and a FLAG-tag. Amp represents an ampicillin resistance gene. The cloning sites and promoter sequences for pRGE3 (B) and pRGE6 (C) are shown at the bottom. The designed DNA oligonucleotides duplex can be inserted into Bsa I sites in pRGE vectors and fused with gRNA scaffold to construct engineered gRNA. The sequence in grey will be replaced by designed DNA sequence encoding gRNA. Italic low case letter indicates overhang sequence after Bsa I digestion.

FIG. 3(A-B) shows a diagram of pRGEB3 (A) and pRGEB6 (B) binary vectors for the Agrobacterium-mediated transient expression or stable transformation. The gRNA scaffold/Cas9 cassettes are the same as those of pRGE3 and pRGE6, but are inserted into the T-DNA region in the pCAMBIA 1300 binary vector.

FIG. 4 shows the pRGE31 and pRGEB31 vectors, which are the modified and improved versions of pRGE3 and pRGEB3, respectively, to facilitate cloning and genome editing in plants according to an exemplary embodiment of the invention.

FIG. 5(A-D) shows the pRGE32 and pRGEB32 vectors for targeted mutation and genome editing in plants according to an exemplary embodiment of the invention. (A and B) The pRGE32 and pRGEB32 vectors incorporate the novel OsUBI10 promoter (Pro_UBI10; SEQ ID NO:1). (C) The OsUBI10 promoter fragment was amplified from 1716 by before the translational start codon. (D) The Cas9 protein expression of pRGE32 is about 5 times higher than that of pRGE31. The Cas9 protein expression was detected by western blotting using Anti-FLAG antibody.

FIG. 6(A-B) provides a diagram for the targeting strategy according to an exemplary embodiment of the invention. (A) Schematic description of rice OsMPK5 locus. The rectangles represent exons, of which black ones indicate the OsMPK5 coding region. The sites targeted by engineered gRNA (PS1-3) are shown as PS1, PS2 and PS3. PSI contains a Kpn I site and PS3 contains a Sac I site. F-256 and R-611 indicate the position of primers used to amplify genomic fragment of OsMPK5. (B) Base pairing between the engineered gRNAs and the targeted sites at the OsMPK5 genomic DNA. PS1-gRNA was paired with the coding strand of OsMPK5 whereas PS2 and PS3 were paired with the template strand of OsMPK5. The predicted gRNA-Cas9 cutting position was indicated with the scissor symbol.

FIG. 7 shows expression of GFP in rice protoplasts. Rice protoplasts were transfected with a plasmid carrying 35S::GFP and observed with a fluorescence microscope at 18, 36 and 60 hours after transfection. The un-transfected protoplasts were red due to auto-fluorescence of chlorophyll.

FIG. 8 shows expression of Cas9 protein in rice protoplasts transfected with the pRGE vector (Vec) or engineered gRNA constructs (PS1-PS3) that targeted OsMPK5. Rice protoplast expressing GFP was used as negative control (CK). Total proteins were extracted from rice protoplasts and the Cas9 fusion protein was detected with an anti-FLAG antibody. The protein loading was shown based on the Coomassie Brilliant Blue staining.

FIG. 9 shows the procedure for restriction enzyme digestion suppressed PCR (RE-PCR) to detect genomic mutation. RE, restriction enzyme.

FIG. 10 shows detection of gene targeting and specific mutations at the PS1 and PS3 sites in the OsMPK5 locus. (A) Detection of mutated genomic sequence by RE-PCR. The genomic DNAs were extracted from the transfected rice protoplasts. Upon digestion with Kpn I or Sac, amplicons could be produced by PCR only when the gene targeting at PS1 and PS3 resulted in mutations at the Kpn I or Sac I site. An amplicon of OsUBQ10 without Kpn I or Sac I in it was used as the control. The relative amount of mutated DNAs in PS1 and PS3 samples was quantified by qPCR and shown in the bottom. (B) Detection of targeted mutation (deletion or insertion) at the PS1 and PS3 sites in the OsMPK5 locus based on DNA sequencing. (C) Targeted mutations revealed by the mismatch-sensitive T7 endonuclease I (T7E1) assay. The DNA fragments were amplified by PCR from genomic DNAs extracted from transfected protoplasts (Vector [Vec] and PS1-3). Mismatches resulting from deletion or insertion at PS1, PS2 and PS3 sites in the OsMPK5 amplicons were detected by T7E1 digestion. Arrows indicate the digested fragments by T7E1. The ratio of cleaved DNA band and total DNA was shown at the bottom.

FIG. 11(A-B) shows chromatographs of Sanger sequencing. Sequencing data reveal deletion or insertion introduced at the PS1 and PS3 sites in the OsMPK5 locus.

FIG. 12 shows homologous sequences in rice genome identified by BLASTN search using PS3-PAM sequence as query. A total of 11 sites in rice genome show similarities to query sequence with expect value less than 100. Among those sites, 7 of them have PAM (highlighted in red) follow the base-pairing region, and might be the potential targets of PS3-gRNA-Cas9.

FIG. 13 shows detection of off-targets caused by PS3-gRNA-Cas9 in rice genome. (A) Base-pairing between PS3-gRNA seed and three potential off-targeted sites. DNA sequence of PAM was indicated in red. The mis-match between gRNA seed and genomic DNA was labeled with circle. The relative position of mis-matches to PAM was shown on the right. (B) Detection of PS3-gRNA-Cas9 editing at the potential off-target sites by RE-PCR. After Sad digestion of genomic DNAs, the PCR product was amplified only from the Chr12-Off-Target site.

FIG. 14(A-D) shows targeted mutations of OsMPK5 detected in stable transgenic rice plants. (A) Vector control plant and two representative transgenic lines (TG4 and TG5) expressing the PS1-gRNA/Cas9 and PS3-gRNA/Cas9, respectively. (B) PCR-T7E1 assay to detect targeted mutation of OsMPK5 in TG4 and TG5 lines. (C) PCR-RE assay to detect mutation at TG4 and TG5 lines. The mutated OsMPK5 is resistant to KpnI (TG4 lines) or Sac I (TG5 lines) digestion. The assay suggests that TG4 #2 is monoallelic mutation whereas TG4 #1, TG5 #1 and TG5 #3 are bioallelic mutation. (D) Mutation revealed by Sanger sequencing of PCR products from TG4-#1 and TG5-#3.

FIG. 15(A-C) shows a diagram of pStGE3 (A) and pStGEB3 (B) vectors for transient and stable transformation of dicot plants such as potato and Arabidopsis. (A) Diagram of pStGE3 vector for transient or stable transformation via protoplast transfection or biolistic bombardment. A DNA-dependent RNA polymerase III (Pol III) U3 promoter from Arabidopsis and Pol III terminator are used to control the transcription of engineered gRNA. 35S promoter and Pol II terminator are used to control the expression of a chimeric Cas9 nuclease fused with 3× FLAG tag. hSpCas9 encodes a human codon optimized Cas9 nuclease which includes a nuclear localization signal (NLS) and a FLAG-tag. Amp represents an ampicillin resistance gene. (B) Diagram of pStGEB3 binary vector for the Agrobacterium-mediated transformation. The gRNA scaffold and Cas9 cassettes are the same as those of pStGE3, but are inserted into the T-DNA region in the pCAMBIA 1300 binary vector. (C) The cloning site and the promoter sequence in pStGE3 are shown. The designed DNA oligonucleotides duplex can be inserted into Bsa I sites and fused with gRNA scaffold to construct engineered gRNA.

FIG. 16(A-B) shows a schematic of targeting the StAS1 locus in potato (Solanum tuberosum) according to an exemplary embodiment of the invention. (A) The rectangles represent exons, of which the numbers show the length of exons and introns. The targeted sites by engineered gRNAs (PS1, PS2) were shown as PS1 and PS2. PS1 contains an SspI site and PS2 contains a XhoI site. AS1-F and AS1-R indicate the position of primers used to amplify genomic fragment of StAS1. (B) Base pairing between the engineered gRNAs and the targeted sites at the StAS1 genomic DNA. PS1-gRNA was paired with the coding strand of StAS1 whereas PS2 was paired with the template strand of StAS1. The predicted gRNA-Cas9 cutting position was indicated with the lightning symbol.

FIG. 17(A-B) shows isolation and transient transformation of potato protoplasts. (A) Expression of GFP in the potato protoplasts from cultivar DM. Potato protoplasts were transfected with a plasmid carrying 35S:: GFP and observed with a fluorescence microscope at 24 hours after transfection. (B) Expression of Cas9 protein in potato protoplasts transfected with the pStGE3 vector. Total proteins were extracted from potato protoplasts transfected with pStGE3 vector and a positive control vector carrying a FLAG tagged fungal MoNLP1 gene, respectively. The Cas9 fusion protein shown in the immunoblot was detected with an anti-FLAG antibody.

FIG. 18(A-C) shows detection of specific mutations at the PS1 and PS2 sites in the StAS1 locus. (A) The genomic DNAs were extracted from the transfected Solanum tuberosum protoplasts. Upon digestion with SspI or XhoI, amplicons could be produced by PCR only when the gene targeting at PS1 and PS2 resulted in mutations at the SspI or XhoI site. (B) The PCR fragments were amplified with a pair of primers (As 1-F and As-R) using genomic DNAs from the transfected Solanum tuberosum protoplasts. The amplicons were then digested with SspI or XhoI. Targeted mutation of PS1 and PS2 sites were detected as un-digestable DNA fragments. (C) Detection of specific mutations (deletion or insertion) at the PS1 and PS2 sites in the StAS1 locus based on DNA sequencing.

FIG. 19(A-B) shows a schematic of targeting the AtPDS3 locus in Arabadopsis thaliana according to an exemplary embodiment of the invention. (A) Schematic description of Arabidopsis AtPDS3 locus. The rectangles represent exons, of which black ones indicate the AtPDS3 coding region. The targeted sites by engineered gRNA were shown as PS1 and PS2. (B) Base pairing between the engineered gRNAs and the targeted sites of the AtPDS3. The predicted gRNA-Cas9 cutting position was indicated with the scissor symbol. The PAM is boxed on both sites.

FIG. 20(A-D) shows targeted mutagenesis at the PS1 site in the AtPDS3 locus. (A) Detection of targeted mutation by RE-PCR. Genomic DNAs were extracted from the wildtype Arabidopsis ecotype Columbia (Col) and individual transgenic lines. Upon digestion with NcoI, amplicons could be produced by PCR only when the genome editing resulted in a mutation and destruction of the NcoI site. (B) Detection of targeted mutation by PCR-RE. The PCR reaction was performed using the genomic DNAs with a pair of specific primers (PDS3-F and PDS3-R). The amplicons were then digested with NcoI, Targeted mutation by the PS1-gRNA/Cas9 construct would destroy the NcoI site and resulted in un-digested bands. (C) Verification of targeted mutation (1-7 by deletion) at the PS1 site of AtPDS3 by DNA sequencing. After NcoI digestion, DNA fragments produced via RE-PCR were cloned into pGEM-T vector and then sequenced. (D) Phenotypic comparison of wildtype (CK) and three AtPDS3 mutants (PS1-9, PS1-11 and PS1-21) at 12 days after germination. The AtPDS3 mutants exhibited reduced plant growth.

FIG. 21(A-B) provides a diagrammatic representation of genome-wide prediction of specific gRNA spacers and assessment of off-target constraints for CRISPR—Cas9 in eight plant species, according to an exemplary embodiment of the invention. (A) Diagrammatic illustration of targeted DNA cleavage by gRNA-Cas9. A gRNA consists of a 5′-end spacer sequence paired to target DNA protospacer and the conserved scaffold (red lines). PAM, protospacer-adjacent motif. (B) A simplified scheme for genome-wide prediction of specific gRNA spacers (see Example IV and FIG. 23 for details). Class 0.0 and Class1.0 gRNA spacers are considered most specific for RGE.

FIG. 22(A-B) shows positive correlation between genome size and (A) NGG—PAM number in eight plant species; and between genome size and (B) the number of specific gRNA spacers was found in eudicots but not in monocots of the grass family. The linear regressed trend line in (B) is shown in grey for eudicots and black for monocots.

FIG. 23 shows percentage of annotated transcript units that could be targeted by specific gRNAs. Eudicots: At, Arabidopsis thaliana; Mt, Medicago truncatula; Sl, Solanum lycopersicum; Gm, Glycine max. Monocots: Bd, Brachypodium distachyon; Os, Oryza sativa; Sb, Sorghum bicolor; Zm, Zea mays.

FIG. 24 shows a flow chart of the analysis pipeline. A genomic segment of rice was used as example for gRNA spacer sequence extraction. The short line labeled the PAM in both strands of the chromosome (black, plus strand; grey, minus strand). As shown in the example, some spacer sequences with 1-3 mismatches would be extracted from the same genome region with consecutive PAM; they could not be considered as off-target and were removed in alignment results. GG_spacer, spacer sequence for NGG-PAM; AG_spacer, spacer sequence for NAG-PAM; minMM, minimal mismatch (including both gaps and substitutions) number of all alignments for each candidate.

FIG. 25 shows per-transcript unit (TU) count of specific gRNA targetable sites in eight plant species. The histogram plots show the distribution of TUs according to their specific gRNAs (Class0.0 and Class1.0) targetable sites. A few of TUs with more than 500 specific gRNA spacers were not shown here.

FIG. 26(A-B) shows identification and design of specific gRNAs using CRISPR-PLANT. All analysis results could be accessed by searching interesting region or genes (A) or viewed in genome browse with JBrowse interface (B). (A) Partial searching and analysis results of Arabidopsis AT1G01010 were shown as an example. (B) Exploring gRNA spacer information of rice OsMPK5 using genome browser in CRISPR-PLANT.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

### Definitions

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989; 3d ed., 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination there between, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possesses catalytic activity for DNA cleavage. A cleavage domain can be contained in a single polypeptide chain or cleavage activity can result from the association of two (or more) polypeptides.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

As used herein, an “enriched” polynucleotide means that a polynucleotide constitutes a significantly higher fraction of the total DNA or RNA present in a mixture of interest than in cells from which the sequence was taken. A person skilled in the art could enrich a polynucleotide by preferentially reducing the amount of other polynucleotides present, or preferentially increasing the amount of the specific polynucleotide, or both. However, polynucleotide enrichment does not imply that there is no other DNA or RNA present, the term only indicates that the relative amount of the sequence of interest has been significantly increased. The term “significantly” qualifies “increased” to indicate that the level of increase is useful to the person using the polynucleotide, and generally means an increase relative to other nucleic acids of at least 2 fold, or more preferably at least 5 to 10 fold or more. The term also does not imply that there is no polynucleotide from other sources. Other polynucleotides may, for example, include DNA from a bacterial genome, or a cloning vector.

As used herein, an “enriched” polypeptide defines a specific amino acid sequence constituting a significantly higher fraction of the total of amino acids present in a mixture of interest than in cells from which the polypeptide was separated. A person skilled in the art can preferentially reduce the amount of other amino acid sequences present, or preferentially increase the amount of specific amino acid sequences of interest, or both. However, the term “enriched” does not imply that there are no other amino acid sequences present. Enriched simply means the relative amount of the sequence of interest has been significantly increased. The term “significant” indicates that the level of increase is useful to the person making such an increase. The term also means an increase relative to other amino acids of at least 2 fold, or more preferably at least 5 to 10 fold, or even more. The term also does not imply that there are no amino acid sequences from other sources. Other amino acid sequences may, for example, include amino acid sequences from a host organism.

As used herein, an “isolated” substance is one that has been removed from its natural environment, produced using recombinant techniques, or chemically or enzymatically synthesized. For instance, a polypeptide or a polynucleotide can be isolated. A substance may be purified, i.e., is at least 60% free, preferably at least 75% free, and most preferably at least 90% free from other components with which it is naturally associated.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a Cas9 polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide disclosed herein through the use of standard genetic and/or recombinant methodologies well known to one skilled in the art. A polynucleotide disclosed herein may be included in a suitable vector.

As used herein, “genetically modified plant” refers to a plant which has been altered “by the hand of man.” A genetically modified plant includes a plant into which has been introduced an exogenous polynucleotide. Genetically modified plant also refers to a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region.

Conditions that are “suitable” for an event to occur, such as cleavage of a polynucleotide, or “suitable” conditions are conditions that do not prevent such events from occurring. Thus, these conditions permit, enhance, facilitate, and/or are conducive to the event.

As used herein, “in vitro” refers to an artificial environment and to processes or reactions that occur within an artificial environment. In vitro environments can consist of, but are not limited to, test tubes. The term “in vivo” refers to the natural environment (e.g., a cell, including a genetically modified microbe) and to processes or reaction that occur within a natural environment.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

It is very difficult and inefficient to perform gene targeting and genome editing in plants due to the low frequency of homologous recombination. Although ZFN- and TALEN-based technologies have enabled genome editing in plants, there remains a need for more efficient, affordable and simple technologies that can greatly facilitate the functional characterization of plant genes and genetic modification of agricultural crops. The RNA-guided CRISPR-associated nuclease has recently emerged as a new tool for genome editing in mammalian and microbial systems. However, it is unclear if the CRISPR/Cas system is functional in plants and can be exploited for genetic modification of crop species. More importantly, the specificity of CRISPR/Cas system in plant genome editing has not been defined yet. In this invention, a series of pRGE vectors based on the Cas9 nuclease have been created to allow gene targeting and genome editing in the plant system. Methods to compute the engineered gRNA specificity for plant genome editing was developed in the invention. In addition, methods for transient expression and stable integration of the transgenes encoding the gRNA molecule and Cas nuclease were described for the plant system. As a proof of concept, three gRNA sequences were individually cloned into the pRGE3 vector and the resulting gene constructs were introduced into rice protoplasts for specific editing of the OsMPK5 gene in the rice genome. Subsequent PCR amplification, restriction enzyme digestion and DNA sequencing demonstrate that a plant gene or genome sequence (OsMPK5 as an example) can be precisely edited and genetically modified using the provided vectors and methods. Furthermore, a general scheme for genetic modifications of plant and crop species by the RNA-guided genome editing method has been outlined, which includes the approaches for generating non-transgenic, genetically engineered plant cultivars.

With further respect to plants, the polynucleotides and vectors described herein can be used to transform a number of monocotyledonous and dicotyledonous plants and plant cell systems, including dicots such as safflower, alfalfa, soybean, coffee, amaranth, rapeseed (high erucic acid and canola), peanut or sunflower, as well as monocots such as oil palm, sugarcane, banana, sudangrass, com, wheat, rye, barley, oat, rice, millet, or sorghum. Also suitable are gymnosperms such as fir and pine.

Thus, the methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant can be identified and isolated by selecting or screening the engineered cells for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. Polynucleotides that are stably incorporated into plant cells can be introduced into other plants using, for example, standard breeding techniques.

DNA constructs may be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet 16:357-384; Rogers et al (1986) Methods Enzymol. 118:627-641). The Agrobacterium transformation system may also be used to transform, as well as transfer, DNA to monocotyledonous plants and plant cells. See Hernalsteen et al (1984) EMBO J 3:3039-3041; Hooykass-Van Slogteren et al (1984) Nature 311:763-764; Grimsley et al (1987) Nature 325:1677-179; Boulton et al (1989) Plant Mol. Biol. 12:31-40; and Gould et al (1991) Plant Physiol. 95:426-434.

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

The disclosed methods and compositions can be used to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

Nucleic acids introduced into a plant cell can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea. One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene and/or CYP74B activity can be used. Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or western blotting). The transgene may be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

### Plasmid Vectors for Plant Gene Targeting and Genome Editing

According to one aspect of the invention, compositions are provided that allow gene targeting and genome editing in plants. In one aspect, plant-specific RNA-guided Genome Editing vectors are provided. In a preferred embodiment, the vectors include a first regulatory element operable in a plant cell operably linked to at least one nucleotide sequence encoding a CRISPR-Cas system guide RNA that hybridizes with the target sequence; and a second regulatory element operable in a plant cell operably linked to a nucleotide sequence encoding a Type-II CRISPR-associated nuclease. The nucleotide sequence encoding a CRISPR-Cas system guide RNA and the nucleotide sequence encoding a Type-II CRISPR-associated nuclease may be on the same or different vectors of the system. The guide RNA targets the target sequence, and the CRISPR-associated nuclease cleaves the DNA molecule, whereby expression of at least one gene product is altered.

In a preferred embodiment, the vectors include a nucleotide sequence comprising a DNA-dependent RNA polymerase III promoter, wherein said promoter operably linked to a gRNA molecule and a Pol III terminator sequence, wherein said gRNA molecule includes a DNA target sequence; and a nucleotide sequence comprising a DNA-dependent RNA polymerase II promoter operably linked to a nucleic acid sequence encoding a type II CRISPR-associated nuclease. The CRISPR-associated nuclease is preferably a Cas9 protein.

In one embodiment, plasmid vectors are provided for transient expression in plants, plant protoplasts, tissue cultures or plant tissues. In a preferred embodiment the vector pRGE3 (SEQ ID NO:2), pRGE6 (SEQ ID NO:4), pRGE31 (SEQ ID NO:6), or pRGE32 (SEQ ID NO:8). In another preferred embodiment, the vector may be optimized for use in a particular plant type or species. In a preferred embodiment, the vector is pStGE3 (SEQ ID NO:10).

In another embodiment, vectors are provided for the Agrobacterium-mediated transient expression or stable transformation in tissue cultures or plant tissues. In particular the plasmid vectors for transient expression in plants, plant protoplasts, tissue cultures or plant tissues contain: (1) a DNA-dependent RNA polymerase III (Pol III) promoter (for example, rice snoRNA U3 or U6 promoter) to control the expression of engineered gRNA molecules in the plant cell, where the transcription was terminated by a Pol III terminator (Pol III Term), (2) a DNA-dependent RNA polymerase II (Pol II) promoter (e. g., 35S promoter) to control the expression of Cas9 protein; (3) a multiple cloning site (MCS) located between the Pol III promoter and gRNA scaffold, which is used to insert a 15-30 by DNA sequence for producing an engineered gRNA. To facilitate the Agrobacterium-mediated transformation, binary vectors are provided, wherein gRNA scaffold/Cas9 cassettes from the plant transient expression plasmid vectors are inserted into a Agrobacterium transformation, for example the pCAMBIA 1300 vector. To program gRNA, a 15-30 by long synthetic DNA sequence complementary to the targeted genome sequence can be inserted into the MCS site of the vector. In a preferred embodiment, the vector for stable transformation of the plant is pRGEB3 (SEQ ID NO:3), pRGEB6 (SEQ ID NO:5), pRGEB31 (SEQ ID NO:7), pRGEB32 (SEQ ID NO:9), or pStGEB3 (SEQ ID NO:11).

Methods to Introduce Engineered gRNA-Cas9 Constructs into Plant Cells for Genome Editing and Genetic Modification.

According to another aspect of the invention, gene constructs carrying gRNA-Cas9 nuclease can be introduced into plant cells by various methods, which include but are not limited to PEG- or electroporation-mediated protoplast transformation, tissue culture or plant tissue transformation by biolistic bombardment, or the Agrobacterium-mediated transient and stable transformation. In one embodiment, rice protoplasts can be efficiently transformed with a plasmid construct carrying a gRNA-Cas9 nuclease specific for a selected target sequence. The transformation can be transient or stable transformation.

Target gene sequences for genome editing and genetic modification can be selected using methods known in the art, and as described elsewhere in this application. In a preferred embodiment, target sequences are identified that include or are proximal to protospacer adjacent motif (PAM). Once identified, the specific sequence can be targeted by synthesizing a pair of target-specific DNA oligonucleotides with appropriate cloning linkers, and phosphorylating, annealing, and ligating the oligonucleotides into a digested plasmid vector, as described herein. The plasmid vector comprising the target-specific oligonucleotides can then be used for transformation of a plant.

### Novel Plant Promoters for Expression Genes and Gene Products

According to one aspect, the invention provides novel nucleotide sequences for use in driving expression of a gene or gene product of interest. In a preferred embodiment, a novel rice promoter (UBI10, SEQ ID NO:1) is provided. The novel promoter may be used to drive expression of a gene or gene product of interest in a plant, including monocot and dicot plants. According to a preferred embodiment, the promoter may be used to drive expression of a gRNA for targeting of a CRISPR/Cas9 gene editing system.

Methods of Designing Specific gRNAs with Minimal Off-Target Risk

According to one aspect, the invention provides methods to design DNA/RNA sequences that guide Cas9 nuclease to target a desired site at a high specificity. The specificity of engineered gRNA could be calculated by sequence alignment of its spacer sequence with genomic sequence of targeting organism.

### Approaches to Produce Non-Transgenic, Genetically Modified Plants or Crops

Using the aforementioned plasmid vectors and delivery methods, genetically engineered plants can be produced through specific gene targeting and genome editing. In many cases, the resulting genetically modified crops contain no foreign genes and basically are non-transgenic. A DNA sequence encoding gRNA can be designed to specifically target any plant genes or DNA sequences for knock-out or mutation via insertion or deletion through this technology. The ability to efficiently and specifically create targeted mutations in the plant genome greatly facilitates the development of many new crop cultivars with improved or novel agronomic traits. These include, but not limited to, disease resistant crops by targeted mutation of disease susceptibility genes or genes encoding negative regulators (e.g., Mlo gene) of plant defense genes, drought and salt tolerant crops by targeted mutation of genes encoding negative regulators of abiotic stress tolerance, low amylose grains by targeted mutation of Waxy gene, rice or other grains with reduced rancidity by targeted mutation of major lipase genes in aleurone layer, etc. Because the CRISPR/Cas gene constructs are only transiently expressed in plant protoplasts and are not integrated into the genome, genetically modified plants regenerated from protoplasts contain no foreign DNAs and are basically non-transgenic. For plant species or cultivars that can be regenerated from protoplasts, gRNA/Cas constructs can be introduced into the binary vectors, such as, for example, the pRGEB32 and pStGEB3 vectors for the Agrobacterium-mediated transformation as described herein. In the case of such Agrobacterium-mediated transformation, the resulting transgenic crop must be backcrossed with wildtype plants to remove the transgene for producing non-transgenic cultivars. In addition to targeted mutation, the gRNA-Cas construct can be introduced together with a donor DNA construct into plant cells (via protoplast transformation or the Agrobacterium-mediated transformation) to create precise nucleotide alterations (substitution, deletion and insertion) and sequence insertion. In one embodiment, herbicide-tolerant crops can be generated by substitutions of specific nucleotides in plant genes such as those encoding acetolactate synthase (ALS) and protoporphyrinogen oxidase (PPO). In addition to targeted mutation of single genes, gRNA-Cas constructs can be designed to allow targeted mutation of multiple genes, deletion of chromosomal fragment, site-specific integration of transgene, site-directed mutagenesis in vivo, and precise gene replacement or allele swapping in plants. Therefore, the invention has have broad applications in gene discovery and validation, mutational and cisgenic breeding, and hybrid breeding. These applications should facilitate the production of a new generation of genetically modified crops with various improved agronomic traits such as herbicide resistance, disease resistance, abiotic stress tolerance, high yield, and superior quality.

### EXAMPLES

### Example I

### Targeted Mutation of a Mitogen-Activated Protein (MAP) Kinase Gene in Rice

Precise and straightforward methods to edit the plant genome are much needed for functional genomics and crop improvement. The inventors herein provide compositions and methods for genome editing and targeted gene mutation in plants via the CRISPR-Cas9 system. Three guide RNAs (gRNAs) with a 20-22 nt seed (also referred as spacer) region were designed to pair with distinct rice genomic sites which are followed by the protospacer adjacent motif (PAM). The engineered gRNAs were shown to direct the Cas9 nuclease for precise cleavage at the desired sites and introduce mutation (insertion or deletion) by error prone non-homologous end joining DNA repairing. By analyzing the RNA-guided genome editing events, the mutation efficiency at these target sites was estimated to be 3-8%. In addition, off-target effect of an engineered gRNA-Cas9 was found on an imperfectly paired genomic site, but it had lower genome editing efficiency than the perfectly matched site. Further analysis suggests that mis-match position between gRNA seed and target DNA is an important determinant of the gRNA-Cas9 targeting specificity. Our results demonstrate that the CRISPR-Cas system can be exploited as a powerful tool for gene targeting and precise genome editing in plants.

Methodologies for precise genome editing are of great importance to functional characterization of plant genes and genetic improvement of agricultural crops. In contrast to the microbial system, it is very inefficient and difficult to achieve successful gene targeting in plants, largely due to the low frequency of homologous recombination (HR). In recent years, sequence-specific nucleases have been developed to increase the efficiency of gene targeting or genome editing in animals and plants. Among them, zinc finger nucleases (ZFNs) and transcription activator-like effector nucleases (TALENs) are the two most commonly used sequence-specific chimeric proteins. Once the ZFN or TALEN constructs are introduced into and expressed in cells, their programmable DNA binding domains can specifically bind to a corresponding sequence and guide the chimer nuclease (e.g., FokI nuclease) to make a specific DNA strand cleavage. In general, single zinc-finger motif specifically recognizes 3 bp, and engineered zinc-finger with tandem repeats can recognize up to 9-36 bp. However, it is quite tedious and time consuming to screen and identify a desirable ZFN. By contrast, TALEs are derived from plant pathogenic bacteria Xanthomonas and contain 34 amino acid tandem repeats in which repeat-variable diresidues (RVDs) at positions 12 and 13 determine the DNA-binding specificity. As a result, TALENs with 16-24 tandem repeats can specifically recognize 16-24 by genomic sequences and the chimeric nuclease can generate DSBs at specific genomic sites. A pair of ZFNs or TALENs can be introduced to generate double strand breaks (DSBs), which activates the error prone DNA repairing systems to introduce mutation at the DNA break site by nonhomologous end joining (NHEJ) mechanism. DSB also increases the homologous recombination (HR) between chromosomal DNA and foreign donor DNA, which greatly improves the gene targeting efficiency. Both ZFN and TALEN have been used in plant gene targeting and genome editing.

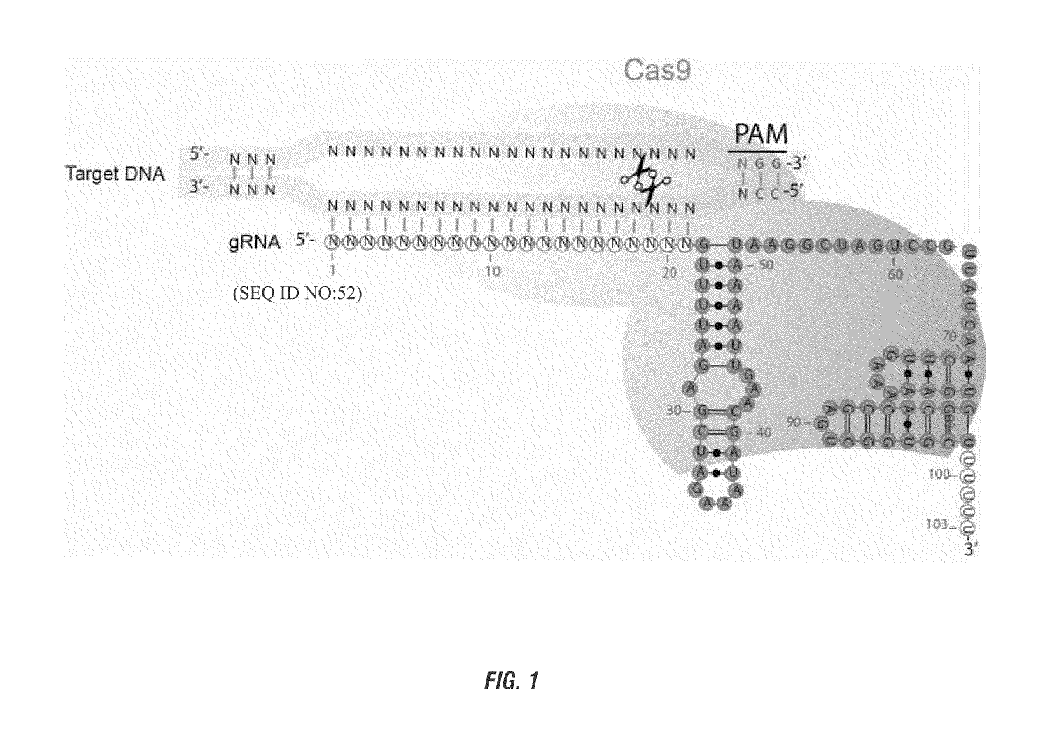

Most recently, a new gene targeting tool has been developed in microbial and mammalian systems based on the cluster regularly interspaced short palindromic repeats (CRISPR)-associated nuclease system. The CRISPR-associated nuclease (Cas) is part of adaptive immunity in bacteria and archaea. The Cas9 endonuclease, a component of Streptococcus pyogenes type II CRISPR-Cas system, forms a complex with two short RNA molecules called CRISPR RNA (crRNA) and transactivating crRNA (transcrRNA), which guide the nuclease to cleave non-self DNA on both strands at a specific site. The crRNA-transcrRNA heteroduplex could be replaced by one chimeric RNA (so-called guide RNA [gRNA]) and the gRNA could be programmed to target specific sites. As shown in FIG. 1, the minimal constrains to program gRNA-Cas9 is at least 15-base-pairing (gRNA seed region) without mistach between the 5′-end of engineered gRNA and targeted genomic site, and an NGG motif (so-called protospacer-adjacent motif or PAM) that follows the base-pairing region in complementary strand of the targeted DNA. The CRISPR/Cas system has been demonstrated for genome editing in human, mice, zebrafish, yeast and bacteria. Due to the significant differences between animals and plants, however, it is important to test the functionality and utility of the CRISPR-Cas system for genome editing and gene targeting in plants.

Here we provide methods and compositions for RNA-guided genome editing in plants using the CRISPR-Cas9 system. As a proof of concept, targeted gene mutation was successfully achieved in three specific sites of a mitogen-activated protein kinase gene in rice genome. Furthermore, the mutation efficiency and off-target effect have been assessed for the RNA-guided genome editing in plants. This study demonstrates that the CRISPR-Cas9 system is functional in plants and can be exploited for gene targeting and genome editing in crop species.

### Results and Discussion

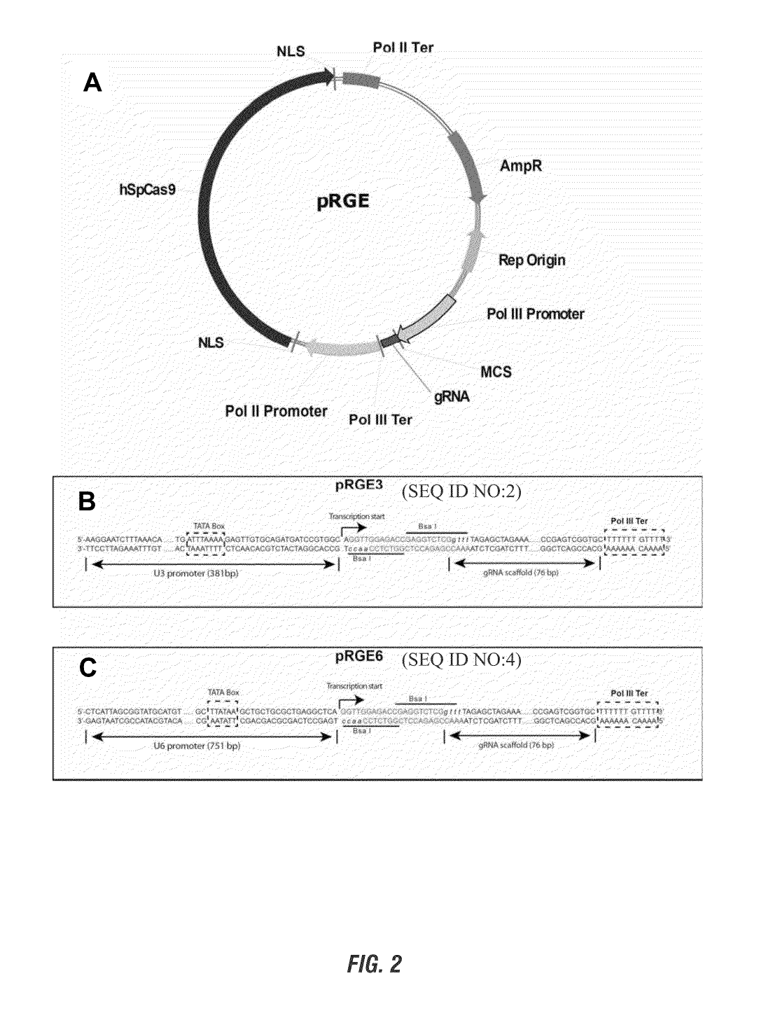

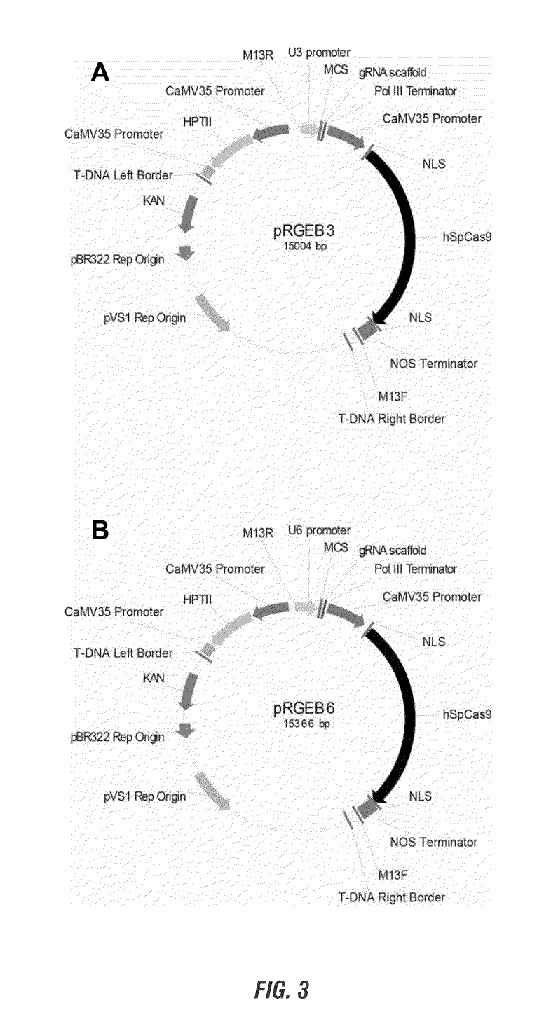

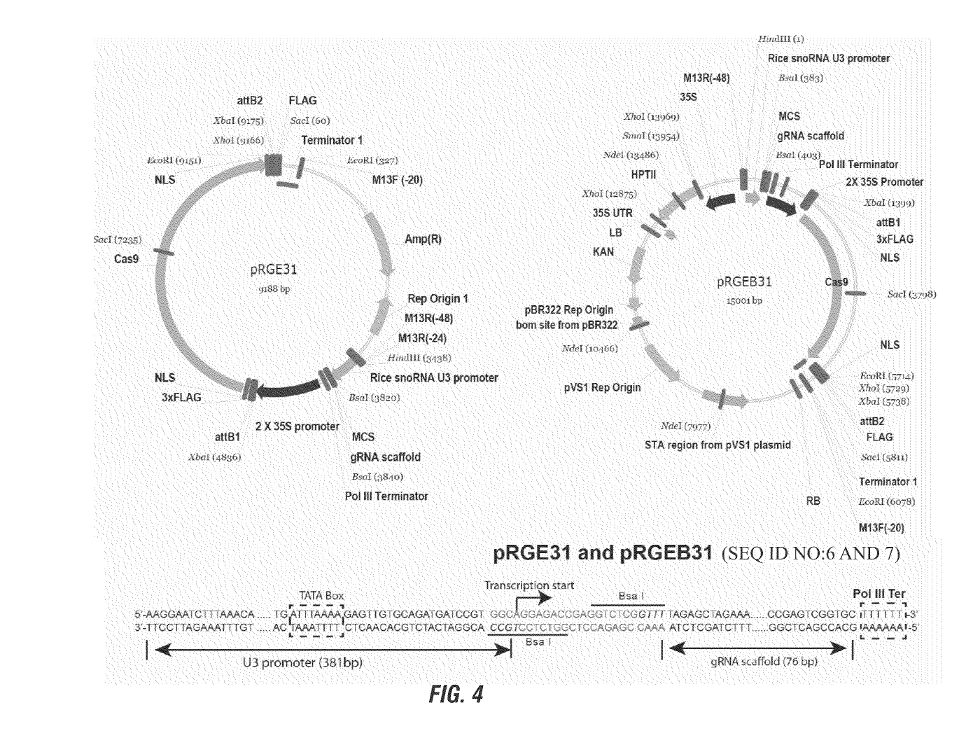

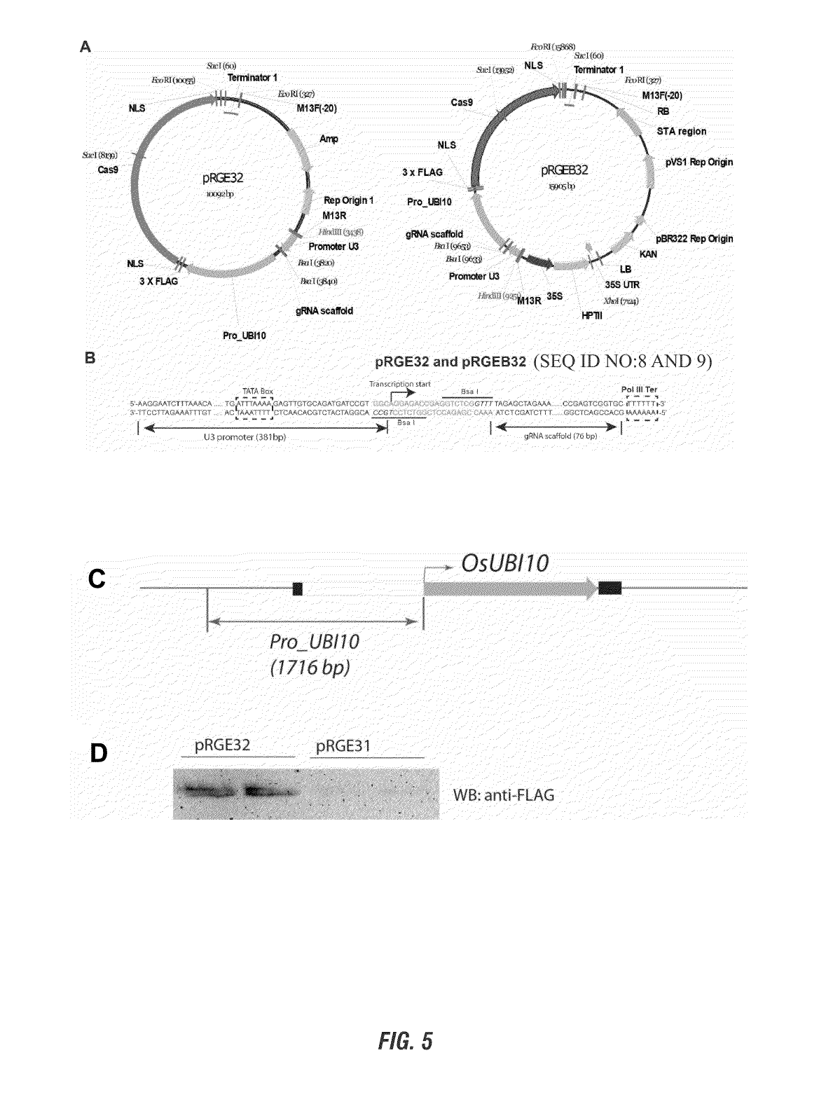

To adapt the CRISPR-Cas9 system for plant genome editing, two RNA-guided Genome Editing vectors (pRGE3 and pRGE6, see FIG. 2) were created for expressing engineered gRNA and Cas9 in plant cells. In both vectors, CaMV 35S promoter was used to control the expression of Cas9 which was fused with a nuclear localization signal and a FLAG tag. As shown in FIG. 2A, the pRGE3 and pRGE6 vectors contain: (1) a DNA-dependent RNA polymerase III (Pol III) promoter (rice snoRNA U3 or U6 promoter, respectively) to control the expression of engineered gRNA molecules in the plant cell, where the transcription was terminated by a Pol III terminator (Pol III Term); (2) a DNA-dependent RNA polymerase II (Pol II) promoter (e. g., CaMV 35S promoter) to control the expression of Cas9 protein; (3) a multiple cloning site (MCS) located between the Pol III promoter and gRNA scaffold (FIGS. 2B and 2C), which is used to insert a 15-30 by DNA sequence as gRNA seed for producing an engineered gRNA. For the Agrobacterium tumefaciens-mediated transformation, the gRNA-Cas9 cassettes from pRGE3 and pRGE6 were inserted into the T-DNA region of pCambia 1300 vector, respectively, to produce pRGEB3 and pRGEB6 (see FIG. 3). In addition, improved versions of plasmid vectors were created for both transient and stable transformation (see FIG. 4 and FIG. 5).

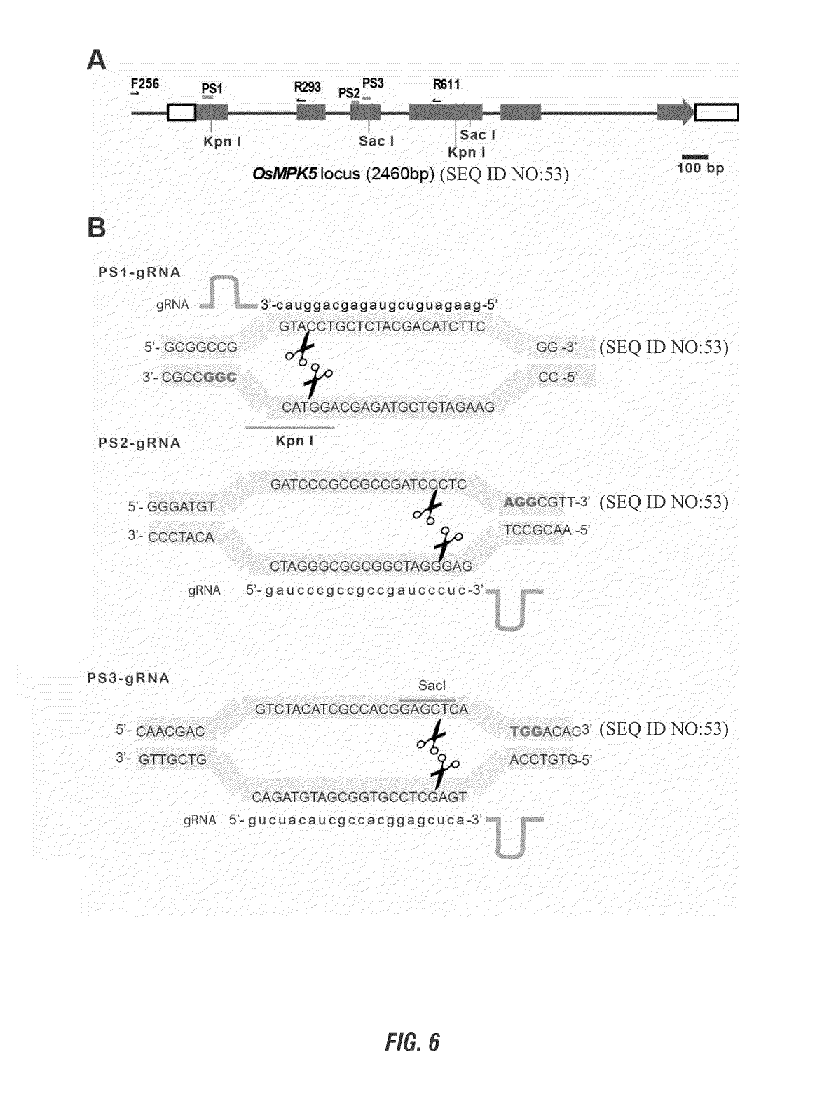

To demonstrate RNA-guided genome editing in plants, the OsMPK5 gene which encodes a stress-responsive rice mitogen-activated protein kinase was chosen for targeted mutation by the CRISPR-Cas9 system. Three guide RNA (gRNA) sequences were designed based on the corresponding target sites in the OsMPK5 locus (PS1, PS2 and PS3, FIG. 6A). The PS1-gRNA seed region (22 nt) was predicted to pair with the template strand of OsMPK5, and would guide Cas9 to make DSB at a Kpn I site. The PS2- and PS3-gRNA seeds region (20 and 22 nt, respectively) were predicted to pair with the coding strand of OsMPK5, and PS3-gRNA would guide Cas9 to make DSB at a Sac I site (FIG. 6B). Subsequently, three gRNA-Cas9 constructs were made by inserting the synthetic DNA oligonucleotides which encode the gRNA seed into the pRGE3 vector.

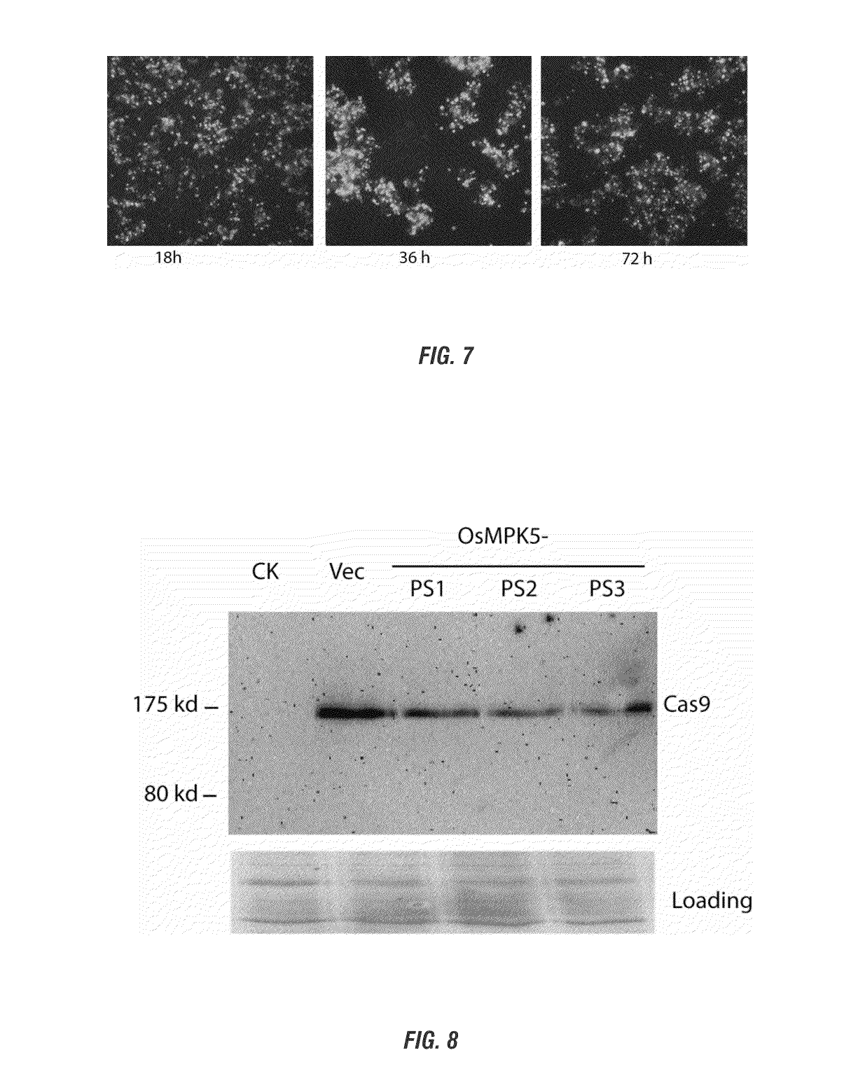

Rice protoplast transient expression system was used to test the engineered gRNA-Cas9 constructs. The efficient transformation of rice protoplasts was demonstrated with a plasmid construct carrying the green fluorescence protein (GFP) marker gene. Fluorescence microscopic analyses indicate that GFP expression was found in approximately 60% of the protoplasts at 18 hours after transformation and in about 90% of the protoplasts at 36-72 hours after transformation (FIG. 7). Following the transformation of empty pRGE3 vector and the pRGE3-PS1/2/3 gRNA constructs into rice protoplasts, the Cas9 nuclease was successfully expressed as revealed by the immunoblot analysis (FIG. 8).

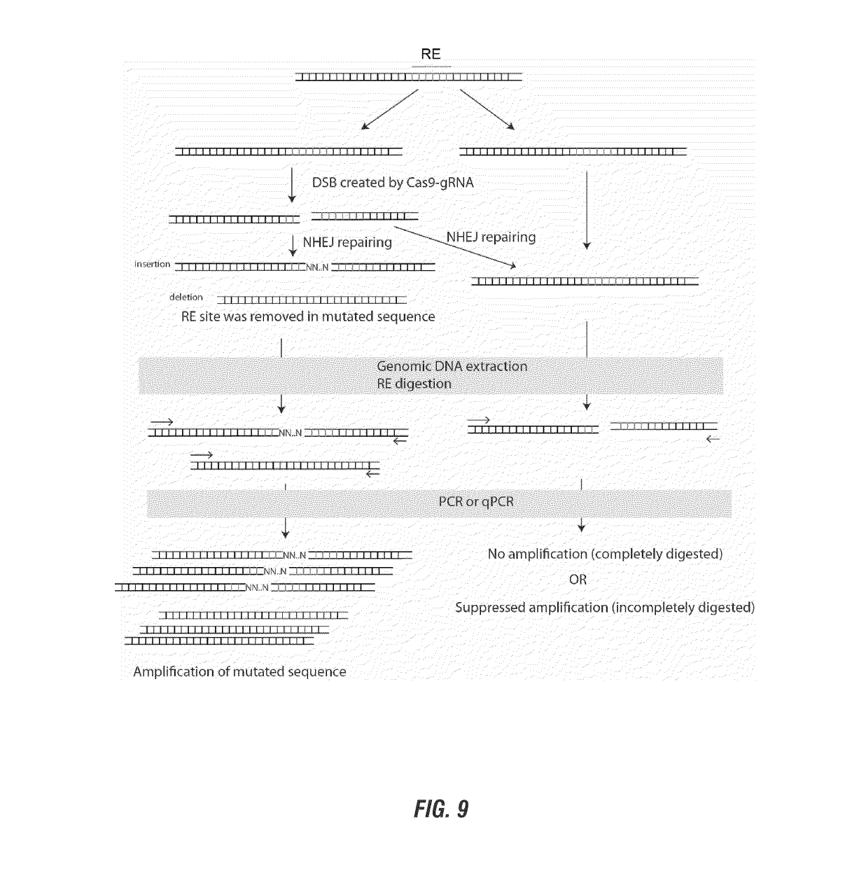

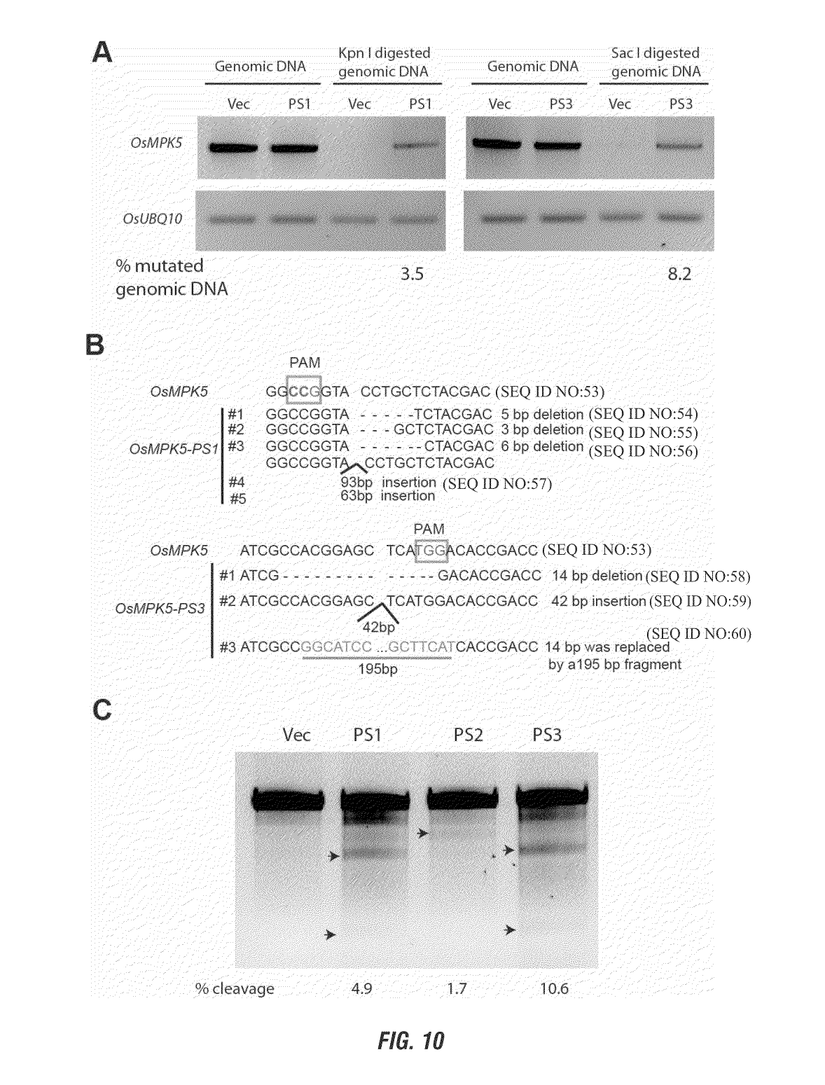

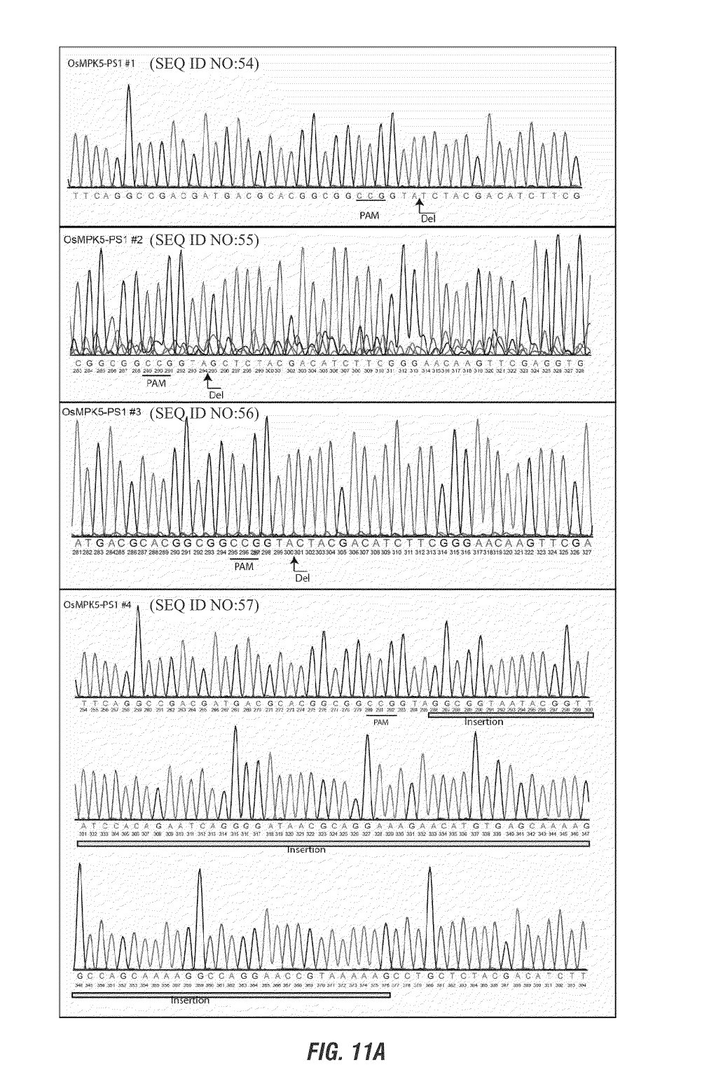

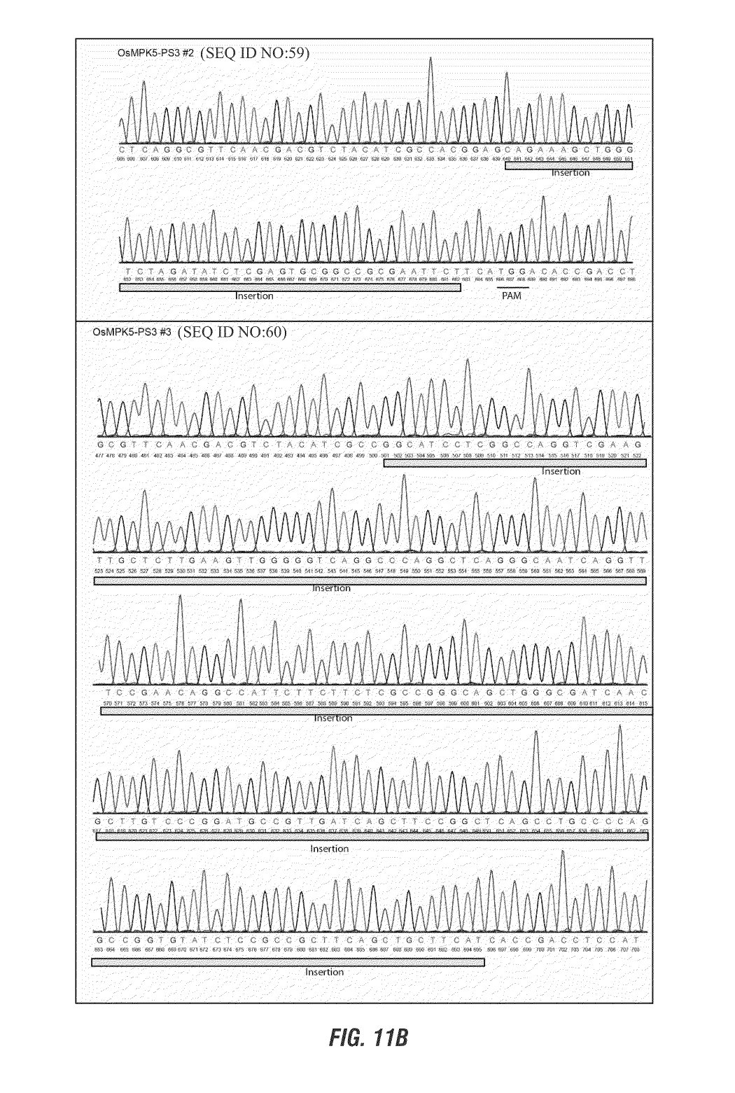

To detect the gRNA-Cas9 mediated precise genome editing, a restriction enzyme digestion suppressed PCR (RE-PCR) was performed to investigate NHEJ introduced mutations in rice genome (FIG. 9). In RE-PCR, plant genomic DNA was first digested with RE whose recognition sequence contains a gRNA-Cas9 cleavage site. A pair of primers (OsMPK5-F256 and OsMPK5-R611) was then used to amplify the targeted region from the digested genomic DNAs (FIG. 9). Because NHEJ introduced mutation will destroy the RE site, amplification of the wild type DNA will be dismissed or suppressed, and mutated sequences will be enriched in PCR products (FIG. 9). Using this method, the expected PCR fragment was amplified from KpnI- or Sac I-digested genomic DNAs extracted from rice protoplasts transformed with pRGE3-PS1 gRNA or pRGE3-PS3 gRNA construct (FIG. 10A), respectively; while no amplification was detected in the sample transformed with the empty vector control. These data suggest that targeted mutations were introduced to the PS1 and PS3 sites, which destroyed the Kpn I and Sac I sites in the OsMPK5 locus. Sanger sequencing of the cloned PCR products further confirmed that targeted mutations were introduced at the predicted Cas9 cleavage site, which is 3 by upstream of PAM (FIG. 10B, FIG. 11). Various mutations, including deletion, insertion or deletion-accompanied insertion were found at both PS1 and PS3 sites. The ratio of deletion to insertion is approximately 1:1; however, the size of deletion is 3-14 by whereas the size of insertion is 42-195 by (FIG. 10B). These results demonstrate that the engineered gRNA-Cas9 can precisely generate DSB at specific sites of the plant genome, leading to targeted gene mutations introduced by the NHEJ DNA repairing machinery.

To estimate the efficiency of genome editing, T7 endonuclease I (T7E1) assay was performed to detect mutation for all three targeted sites in the OsMPK5 locus. In this assay, amplicons encompassing targeted sites were amplified from genomic DNA and treated with mis-match sensitive T7E1 after melting and annealing, and cleaved DNA fragments would be detected if amplified products containing both mutated and wild type DNA. As shown in FIG. 10, T7E1 digested fragments were detected in the PS1/2/3 samples but not in the empty vector control. Based on the ratio of T7E1 digested and undigested DNAs, the percentage of targeted mutations in OsMPK5 was about 4.9%, 1.7% and 10.6% for PS1, PS2, and PS3 samples (FIG. 10C). We also performed RE-qPCR for more accurate estimation of genome editing efficiency at PS1-gRNA and PS3-gRNA targeted sites and obtained the mutation frequencies of 3.5% (PS1) and 8.2% (PS3) (FIG. 10A and Table 2). The relatively minor discrepancy in the mutation frequency detected by the T7E1 and RE-qPCR methods is likely due to the different assay methods and experimental variations. However, both methods indicate that gRNA-Cas9 mediated genome editing efficiency in plants ranges from 3% to 8%, which is in the same range of genome editing efficiency in animal cells.

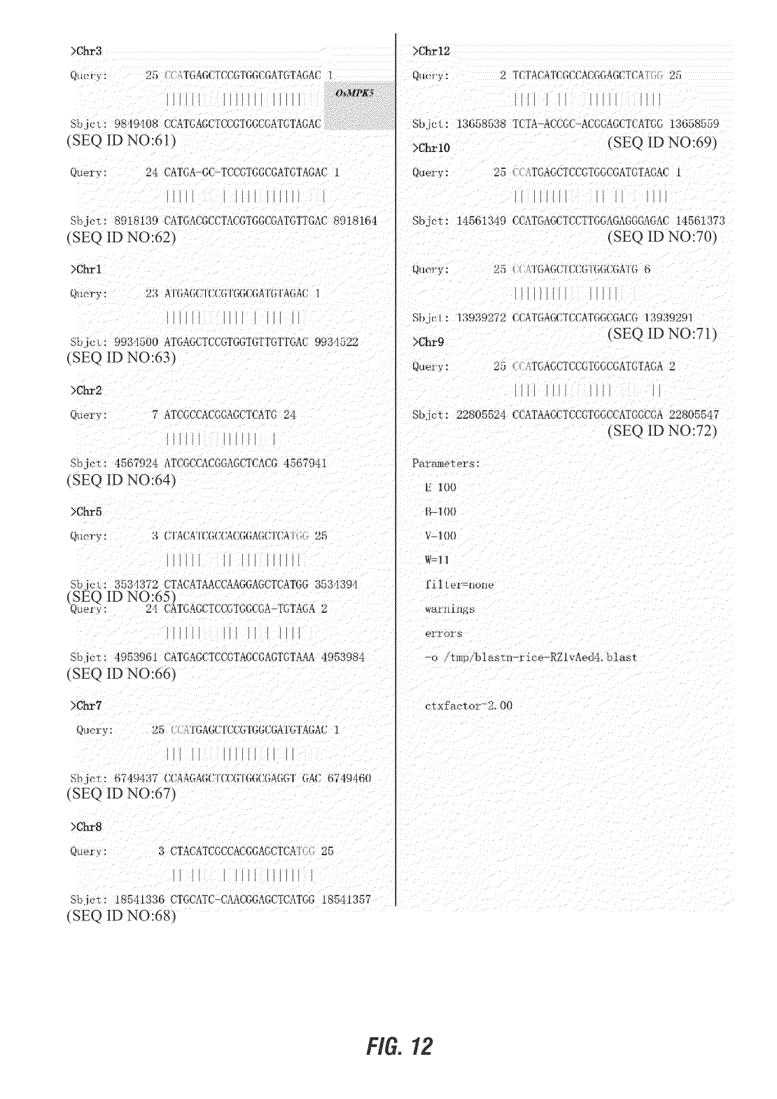

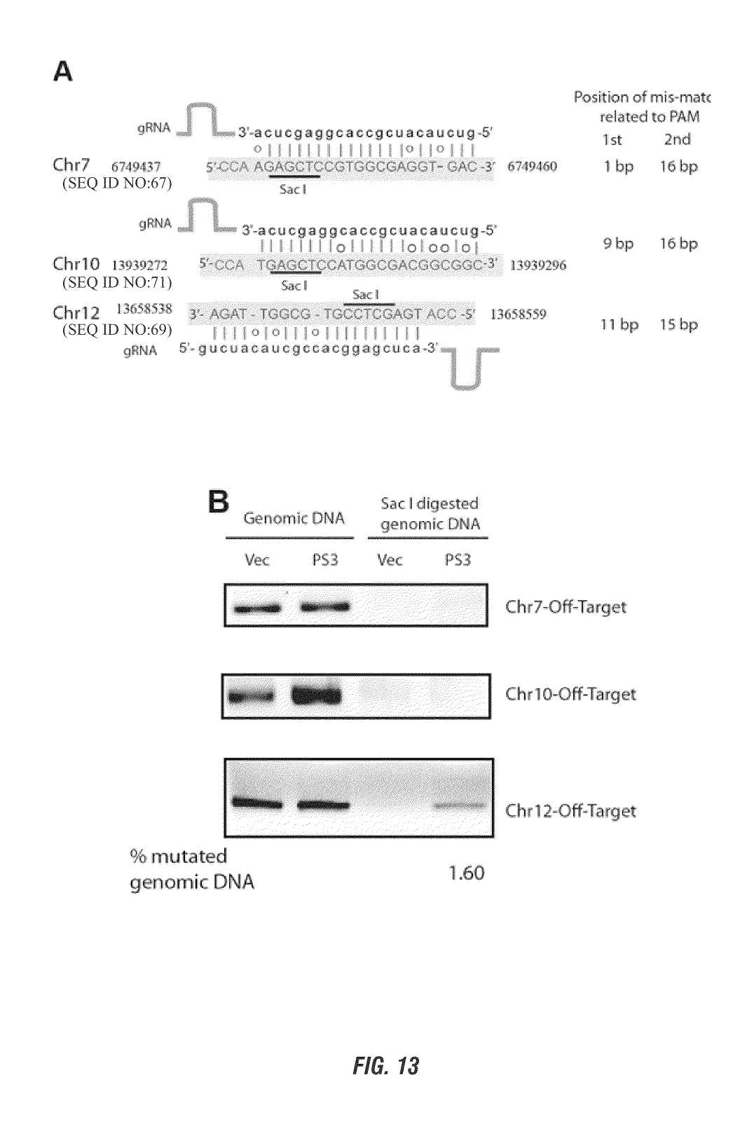

Furthermore, we analyzed the potential off-targets of PS3 gRNA-Cas9 in vivo. After searching the rice genomic sequence using PS3 target sequence with PAM, eleven genomic sites were found to share significant sequence similarity to PS3 sites, and 7 of them contain PAM motif which were potentially targeted by PS3 gRNA-Cas9 (FIG. 12). Based on the mis-match pattern between PS3 gRNA seed sequence and those sites, three genomic sites (Chr7/10/12-Off-Target, FIG. 13A) were selected and analyzed for potential cleavage by PS3 gRNA-Cas9. Because these selected sites also contain a Sac I recognition site covering the potential Cas9 cleavage position, the off-target effect could be tested by RE-PCR. Mutated genomic DNA product was detected by RE-PCR at Chr12-Off-Target site (FIG. 13B), but not in other two sites (Chr7- and Chr10-Off-Target sites). The mutation frequency at Chr12-Off-Target site is about 1.6% (FIG. 13B and Table 2), which is five times lower than that of the OsMPK5 PS3 site. By comparing the mis-match position related to PAM in these three sites, all of them show a single mis-match in the 15 by region proximal to PAM, but the most significant difference between the PS3-gRNA-Cas9 cut and un-cut sites is the position of the first mis-match proximal to PAM which is 1 (Chr7-Off-Target) and 9 (Chr10-Off-Target) in un-cut sites, but is 11 (Chr12-Off-Target) in cut sites (FIG. 13). This is slightly different from human cells in which a single mis-match at 11 by to PAM dismissed the gRNA-Cas9 cleavage (15). Therefore, we speculate that a single mis-match in the 10 by long paring region proximal to PAM will dismiss the gRNA-Cas9 cleavage on non-perfect matched site in plant cells.

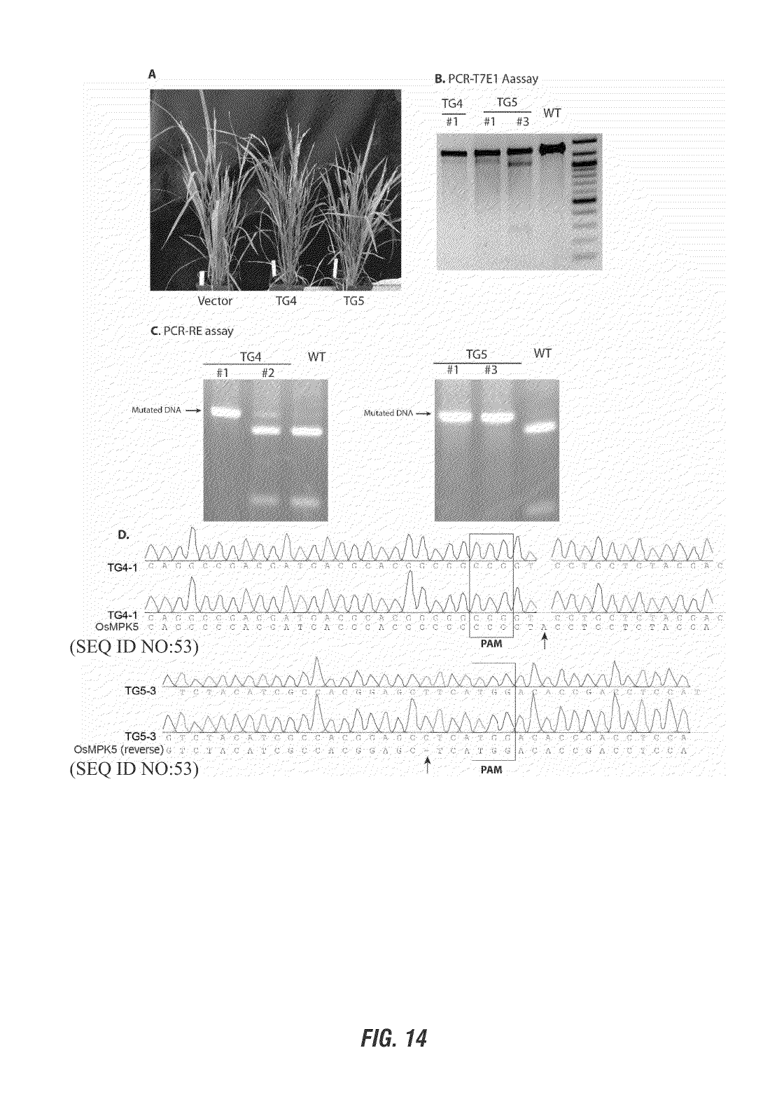

In addition to demonstrating genome editing in rice protoplasts, stable transgenic rice lines were generated expressing gRNA/Cas9 constructs via the Agrobacterium-mediated transformation. The transgenic rice plants expressing PS1-gRNA (TG4 lines) and PS3-gRNA (TG5 lines) were examined by T7E1 assay, PCR-RE assay and Sanger sequencing (FIG. 14). The PCR-RE assay revealed that PCR amplicon from three TO individuals (TG4 #1, and TG5 #1/#3) are resistant to RE digestion, suggesting completely mutated OsMPK5 in these plants (FIG. 14C). The T7E1 assay, which could distinguish heterozygous (monoallelic) from homozygous (i.e. biallelic) mutations, was further performed to examine these T0 individuals. The results show that PCR products from TG4 #1 and TG5 #1 lines are resistant to T7E1 digestion, suggesting they harbored homozyogous mutations on OsMPK5. But PCR amplicons of TG5 #3 was digested by T7E1, suggesting monoallelic mutations of OsMPK5 in this line (FIG. 14B). The T7E1 and PCR-RE assay results was further confirmed by Sanger sequencing of the PCR amplicon from TG4-1 and TG5-3 lines. The sequencing results show that 1 bp insertion/deletion was found at the designed Cas9 cut position (FIG. 14D). These results showed that targeted mutation of OsMPK5 was detected with either biallelic (TG4 line #1 and TG5 line #1) or monoallelic deletion (TG5 line #3) of a single nucleotide, which resulted in the frame-shift and inactivation of OsMPK5. Thus, expression of engineered gRNA and Cas9 in stable transgenic plants would result in heterozygous or homozygous mutations precisely at the targeting sites.

Using rice (a model plant and important crop) as an example, we demonstrated that Cas9 could be guided by engineered gRNA for precise cleavage and editing of the plant genome. Since the specificity of the CRISPR-Cas9 system is based on nucleotide pairing rather than the protein-DNA interaction, this method is likely much simpler, more specific and more effective than the existing ZFN and TALEN systems for genome editing in plants. Besides, the commonly used FokI nuclease domain in TALEN and ZFN requires dimerization to cleave DNA. As a result, a pair of ZFNs or TALENs is needed to make one DSB in genome. In the CRISPR-Cas9 system, only single gRNA is needed to target one genomic site, which is much flexible and easy for multipurpose genome editing. Recent work in mice showed that five genes were destroyed in one step using the CRISPR-Cas9 system, revealing the high capacity of this tool for functional genomic analysis. The short PAM sequence is present in the plant genome at high frequency (for example, 141 PAMs were found in 1110 by coding region of the OsMPK5 gene), suggesting the possibility of targeting and editing of every plant gene using this method. Although we have detected an off-target mutation generated by the PS3-gRNA-Cas9 cleavage (FIG. 13), this is predictable and can be avoid by designing a more specific gRNA sequence that uniquely pairs with a target sequence, especially the 1-10 by region proximal to PAM in target sites. In addition, the frequency for off-target editing at imperfectly paired region was much lower than that of the genuine site (FIG. 13). Even off-target happens in practice, it can be removed by crossing mutants with wild type plants. Therefore, the CRISPR-Cas system can be exploited as a powerful genome editing and gene targeting tool for functional characterization of plant genes and genetic modification of agricultural crops.

### Materials and Methods

Construction of RNA-Guided Genome Editing Vectors for the Plant System

To construct pRGE3 and pRGE6 vectors, rice snoRNA U3 and U6 promoters were amplified from rice cultivar Nipponbare genomic DNA using primer pairs UGW-U3-F/Bsa-U3-R, and UGW-U6-F/Bsa-U6-R, respectively (see Table 1 for the list of primer sequences). The DNA sequence encoding the gRNA scaffold was amplified from the pX330 vector using a pair of primers (Bsa-gRNA-F and UGW-gRNA-R). The PCR product of U3 or U6 promoter and gRNA scaffold was fused by overlapping PCR. The U3 or U6 promoter-gRNA fragment was then cloned into the Hind III site of pUGW11-BsaI vector through the Giboson assembly method to produce pUGW-U3-gRNA and pUGW-U6-gRNA. pUGW11-BsaI was derived from pUGW11 by removing two Bsa I sites in Amp resistance gene and 35S promoter using site-directed mutangenesis (Strategene). The primer sequences used for site-directed mutagenesis were shown in Table 1. The Cas9 gene fragment was cut from pX330 using NcoI and EcoRI and then inserted into pENTR11 (Invitrogen). The Cas9 was subsequently introduced into pUGW-U3-gRNA or pUGW-U6-gRNA by LR reaction (Invitrogen), resulting in the pRGE3 and pRGE6 vector (see FIG. 2). In addition, two binary vectors (pRGEB3 and pRGEB6, see FIG. 3) were made by inserting the gRNA scaffold/Cas9 cassettes from pRGE3 and pRGE6 into the pCAMBIA 1300-BsaI vector. The pCAMBIA 1300-BsaI was derived from pCAMBIA1300 by removing BsaI sites in the 35S promoter using site-directed mutagenesis (Stratagene).

Gene Targeting Constructs for Precise Disruption of the OsMPK5 Gene

DNA sequences encoding gRNAs were designed to target three specific sites in the exons of OsMPK5 (see FIG. 6). For each target site, a pair of DNA oligonucleotides (Table 1) with appropriate cloning linkers were synthesized. Each pair of oligonucleotides were phosphorylated, annealed, and then ligated into Bsa I digested pRGE3 or pRGE6 vectors. After transformation into E. coli DH5-alpha, the resulting constructs were purified with QIAGEN Plasmid Midi kit (Qiagen) for subsequent use in rice protoplast transfection. For stable transformation, DNA oligo which used to construct the PS1-gRNA and PS3-gRNA (Table 1) were inserted into pRGEB3 (FIG. 3). The resulting gene constructs were introduced into the Agrobacterium tumefaciense straint EHA105 via electroporation.

Rice Protoplast Preparation and Transformation

Rice protoplasts were prepared from 10-day-old young seedlings of Nipponbare cultivar (Oryza sativa spp. japonica) after germination in MS media. The protoplasts were isolated by digesting rice sheath strips in Digestion Solution (10 mM MES pH5.7, 0.5 M Mannitol, 1 mM CaCl₂, 5 mM beta-mercaptoethanol, 0.1% BSA, 1.5% Cellulase R10 [Yakult Pharmaceutical, Japan], and 0.75% Macerozume R10 [Yakult Pharmaceutical, Japan]) for 5 hours. After filtering through Nylon mesh (35 um), the protoplasts were collected and incubated in W5 solution (2 mM MES pH5.7, 154 mM NaCl, 5 mM KCl, 125 mM CaCl₂) at room temperature (25° C.) for 1 hour. The W5 solution was then removed by centrifugation at 300×g for 5 min, and rice protoplasts were resuspended in MMG solution (4 mM MES, 0.6 M Mannitol, 15 mM MgCl2) to a final concentration of 1.0×10⁷/ml. For transformation, 10 ul of plasmids (5-10 ug) was gently mixed with 100 ul of protoplasts and 110 ul of PEG-CaCl₂ solution (0.6 M Mannitol, 100 mM CaCl₂ and 40% PEG4000), and then incubated at room temperature for 20 min. Transformation was stopped by adding 2× volume of W5 solution. Transformed protoplasts were then collected by centrifugation and resuspended in WI solution (4 mM MES pH5.7, 0.6 M Mannitol, 4 mM KCl). The transformed protoplasts were maintained in 24-well culture plates. After 24-72 hours of incubation in WI solution, protoplasts were collected by centrifugation at 300×g for 2 min and frozen in -80° C.

Agrobacterium-Mediated Rice Transformation

Embryogenic calli derived from seeds of Nipponbare cultivar were used for the Agrobacterium-mediated stable transformation according to the previously described methods (Xiong and Yang, 2003).

Immunoblot Analysis

To extract total proteins, 100 ul of Lysis Buffer (25 mM Tris-HCl pH7.5, 150 mM NaCl, 2% Triton X-100, 10% glycerol, 5 ug/mL protease inhibitor cocktail [Sigma-Aldrich]) was added to 1×10⁶ rice protoplasts. The cell debris was removed by centrifugation at 13000×g for 10 min. 10 ul of protein extract was separated by 10% SDS-PAGE and transferred to PVDF membrane. The Cas9-FLAG fusion protein was detected with the anti-FLAG antibody (Sigma-Aldrich).

Genomic DNA Extraction

Genomic DNA was extracted from rice protoplasts or seedling leaves by adding 100 ul of pre-heated CTAB buffer and incubated at 65° C. for 20 min. 40 ul of chloroform was then added; the resulting mixtures were incubated at room temperature (25° C.) in a end-to-top rocker for 20 min. After centrifugation at 16000×g for 5 min, the supernatant was transferred to a new tube and mixed with 250 ul of ethanol. Following incubation on ice for 10 min, genomic DNA was precipitated by centrifuge at 16000×g for 10 min at room temperature. The DNA pellet was washed with 0.5 ml of 70% ethanol and air dried. The genomic DNA was then dissolved in 100 ul of dH₂O and its concentration was determined by spectrophotometer.

Detection of Specific Mutations in OsMPK5

Restriction Enzyme Digestion Suppressed PCR

To detect mutation at desired restriction enzyme sites, 500 ng of genomic DNA was digested with Kpn I (Vector and OsMPK5-PS1) or Sac I (Vector and OsMPK5-PS3) at 37° C. for 2 hours. The DNA fragments containing the gRNA-Cas9 target sites were then amplified by PCR (primers sequence in Table 1) from the digested and un-digested genomic DNA using AmpliTaq Go1d360 Master Mix (Life Technologies). The PCR product was analyze by electrophoresis in 1% agrose gel. To identify targeted gene mutation, purified PCR products from RE digested template were cloned to pGEM-T easy vector by TA cloning (Promega), and resulting random colonies were used for plasmid extraction and DNA sequencing.

To determine mutation rate on PS1-and PS3-gRNA targeted sites, quantitative PCR was performed to quantify the amount of mutated genomic DNA. The qPCR was performed in StepOne plus (Life Technologies) using GoTaq qPCR Master Mix (Promega). The calculation of mutated genomic DNA is shown in Table 2.

T7 Exonuclease I Assay

To detect mutation by T7 exonuclease I (T7E1) assay, the DNA fragments containing the targeted sites were amplified from genomic DNA using a pair of primers (OsMPK5-F256 and OsMPK5-R611) and Phusion High-Fidelity DNA Polymerase (NEB). The PCR product was purified using PCR Purification Column (Zymo Research) and concentration was determined with a spectrophotometer. 100 ng of purified PCR product was then denatured-annealed under the following condition: 95° C. for 5 min, ramp down to 25° C. at 0.1 C/sec, and incubate at 25° C. for additional 30 min. Annealed PCR products were then digested with 5U of T7E1 for 2 hours at 37° C. The T7E1 digested product was separated by 1% agrose gel electrophoresis and stained with ethidium bromide. The intensity of DNA bands was calculated using Image J (http://rsbweb.nih.gov/ij/).

Bioinformatic Analysis of Off-Target Sites

To identify potential off-target sites of PS3-gRNA, a 25 by long PS3-gRNA targeted OsMPK5 DNA sequence (included base-pairing region and PAM) was used to search rice genome sequence using BLASTN program in Rice Genome Annotation Project Database (http://rice.plantbiology.msu.edu). For BLASTN, the expect value and word length were set to 100 and 11, respectively (FIG. 12).

Accession Numbers

Sequence data from this article can be found in the EMBL/GenBank data libraries under accession number: OsMPK5 (AF479883), OsUBQ10 (AK101547), pUGW11 (AB626669).

TABLE 1
Oligonucleotides for making plasmid vectors and
OsMPK5 targeting constructs.

| | | |
|---|---|---|
| Purpose | Primer Name | Sequence |

Primers for plasmid construction

| | | |
|---|---|---|
| Rice U6 | UGW-U6-F | 5′- |
| promoter | | GACCATGATTACGCCAAGCTTCTCATTAGCGGT |
| | | ATGCATGTTGG-3′ (SEQ ID NO: 12) |
| | Bsa-U6-R | 5′-CGAGACCTCGGTCTCC |
| | | AACCTGAGCCTCAGCGCAGC-3′ (SEQ ID NO: 13) |
| Rice U3 | UGW-U3-F | 5′- |
| Promoter | | GACCATGATTACGCCAAGCTTAAGGAATCTTTA |
| | | AACATACG-3′ (SEQ ID NO: 14) |
| | Bsa-U3-R | 5′- |
| | | CGAGACCTCGGTCTCCAACCTGCCACGGATCAT |
| | | CTGC-3′ (SEQ ID NO: 15) |
| gRNA | Bsa-gRNA-F | 5′-GGAGACCGAGGTCTCGGTTTTAGAGCTAGAA |
| scaffold | | ATA-3′ (SEQ ID NO: 16) |
| | UGW-gRNA-R | 5′-GGACCTGCAGGCATGCACGCGCTAAAAACGG |
| | | ACTAGC-3′ (SEQ ID NO: 17) |

oligonucleotides for site-directed mutagenesis
to remove Bsa I sites in vectors

| | | |
|---|---|---|
| Remove BsaI | 35S-Mut-F | 5′-GAGAGGCTTACGCAGCAGCACTCATCAAGAC |
| in 35S | | GATCTAC-3′ (SEQ ID NO: 18) |
| Remove BsaI | Amp-Mut-F | 5′-GCCGGTGAGCGTGGCACTCGCGGTATCATT-3′ |
| in Amp gene | | (SEQ ID NO: 19) |

Oligonucleotides used to generate DNA sequences encoding gRNAs

| | | |
|---|---|---|
| OsMPK5-PS3 | OsMPK5PS3-F | 5′-GGTT GTCTACATCGCCACGGAGCTCA-3′ (SEQ |
| | | ID NO: 20) |
| | OsMPK5PS3-R | 5′-AAAC TGAGCTCCGTGGCGATGTAGAC-3′ |
| | | (SEQ ID NO: 21) |
| OsMPK5-PS2 | OsMPK5PS2-F | 5′-GGTT GATCCCGCCGCCGATCCCTC-3′ (SEQ ID |
| | | NO: 22) |
| | OsMPK5PS2-R | 5′-AAAC GAGGGATCGGCGGCGGGATC-3′ (SEQ |
| | | ID NO: 23) |
| OsMPK5-PS1 | OsMPK5PS1-F | 5′-GGTT GAAGATGTCGTAGAGCAGGTAC-3′ |
| | | (SEQ ID NO: 24) |
| | OsMPK5PS1-R | 5′-AAAC GTACCTGCTCTACGACATCTTC-3′ (SEQ |
| | | ID NO: 25) |

Primers used to amplify Cas9-gRNAs targeted sites

| | | |
|---|---|---|
| OsMPK5 | OsMPK5-F2 | 5′-GCCACCTTCCTTCCTCATCCG-3′ (SEQ ID |
| | 56 | NO: 26) |
| | OsMPK5-R6 | 5′-GTTGCTCGGCTTCAGGTCGC-3′ (SEQ ID NO: 27) |
| | 11 | |
| Chr7-off-target | Chr7-PS3-F | 5′-CATCAGGAAGGTTCGCCAGCAC-3′ (SEQ ID |
| | | NO: 28) |
| | Chr7-PS3-R | 5′-ATCATATCTGGGGTCGGATAGAACC-3′ (SEQ ID |
| | | NO: 29) |
| Chr10-off-target | Chr10-PS3-F | 5′-ACAGATTGCCCCAGCGAGAT-3′ (SEQ ID |
| | | NO: 30) |
| | Chr10-PS3-R | 5′-TGTGAGAACCCCGCATCCA-3′ (SEQ ID NO: 31) |
| Chr12-off-target | Chr12-PS3-F | 5′-CTATTTCCGCTGCGAACCAT-3′ (SEQ ID NO: 32) |
| | Chr12-PS3-R | 5′-AGTGACGGCGGGTGCTAGG-3′ (SEQ ID NO: 33) |
| OsUBQ10 | OsUBQ10-F | 5′-TGGTCAGTAATCAGCCAGTTTG-3′ (SEQ ID |
| | | NO: 34) |
| | OsUBQ10-R | 5′-CAAATACTTGACGAACAGAGGC-3′ (SEQ ID |
| | | NO: 35) |

TABLE 2
Relative quantification of mutated genomic DNA using RE-qPCR

| | Genomic | | | | | % of | SD (% of | % of |
|---|---|---|---|---|---|---|---|---|
| Targeted | DNA | ΔCt | ΔCt | | ΔΔCt | undigested | undigested | Mutated |
| Gene | Sample | mean | SD | ΔΔCt | SD | DNA | DNA) | DNA |
| OsMPK5 | Vec | −0.22 | 0.07 | | | | | |
| | PS1 | −0.05 | 0.10 | | | | | |
| | Vec-Kpn I | 8.00 | 0.37 | 8.23 | 0.22 | 0.33%* | 0.02% | |
| | PS1-Kpn I | 4.63 | 0.19 | 4.68 | 0.12 | 3.91% | 0.15% | 3.58% |
| | PS3 | 0.25 | 0.05 | | | | | |
| | Vec-Sac I | 7.36 | 0.16 | 7.58 | 0.10 | 0.52%* | 0.02% | |
| | PS3-Sac I | 3.77 | 0.17 | 3.51 | 0.10 | 8.76% | 0.27% | 8.23% |
| Chr12-Off- | Vec | −0.48 | 0.11 | | | | | |
| Target | PS3 | 0.36 | 0.13 | | | | | |
| | Vec-Sac I | 6.30 | 0.25 | 6.78 | 0.16 | 0.91%* | 0.04% | |
| | PS3-Sac I | 5.67 | 0.05 | 5.32 | 0.08 | 2.51% | 0.06% | 1.60% |

ΔCt = Ct_{targeted gene} − Ct_{OsUBQ10}
ΔΔCt = ΔCt_{Enzyme digested} − ΔCt_{undigested}
[% of undigested DNA] = 2^{-ΔΔCt}
[% of Mutated Genomic DNA] = [% of undested DNA]_{PS} − [% of undigested DNA]_{Vec}
*This number indicates the percentage of genomic DNA not cut by Kpn I or Sac I.
SD, standard deviation (n = 3).

### Example II

### Genome Editing in Potato (a Dicot Food Crop)

The above example demonstrated how CRISPR/Cas9 technology may be adapted and applied to gene editing in monocots and cereal crops such as rice. In this example, the Inventors sought to apply the current genome editing technologies in dicot crops such as potato (Solanum tuberosum), the most important non-grain food crop of the world. The Inventors successfully employed transient expression method to deliver Cas9, along with a synthetic gRNA targeting the StAS1 gene, into potato leaf protoplasts. The expression of Cas9 or gRNA alone did not cause any mutations, and DNA sequencing confirmed that a potato asparagine synthase gene (StAS1) was mutated at the target site in transfected potato protoplasts expressing both Cas9 and gRNA. The mutation rate with the CRISPR/Cas9 system in potato protoplasts was approximately 3.6%-4.6%. This is the first demonstration of genomic editing in potato using CRISPR/Cas9 system, which will promote the study of potato gene functions and genetic improvement.

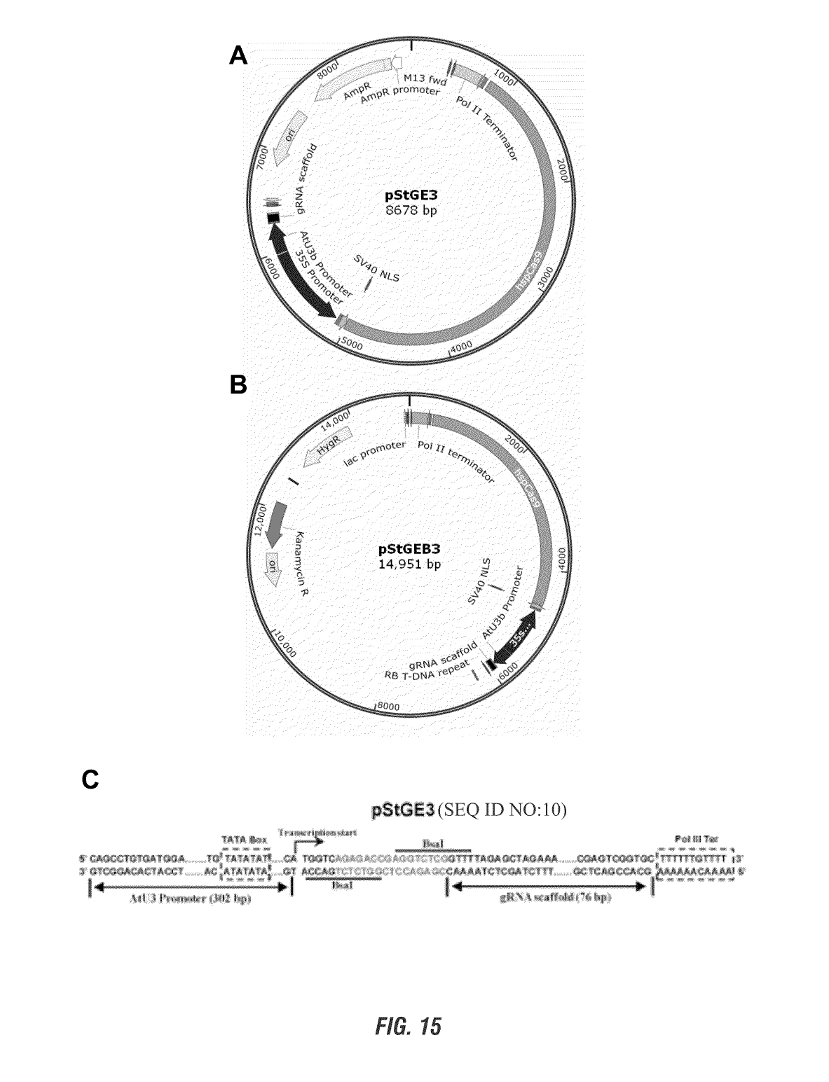

To test the potential of the CRISPR/Cas9 system for targeted mutagensis in potato, transient expression using potato leaf protoplasts was employed to deliver the Cas9 endonuclease and a gRNA. One Solanum tuberosum Genome Editing vector (pStGE3, FIG. 15A) was created to express engineered gRNA targeting a potato gene and Cas9 protein which was fused with a nuclear localization signal and a FLAG tag. As shown in FIG. 15A, the pStGE3 vector contain several important functional elements: (1) a DNA-dependent RNA polymerase III (pol III) promoter (Arabidopsis U3 promoter) to control the expression of engineered gRNA targeting potato genes in the plant cell, where the transcription was terminated by a Pol III terminator (Pol III Term); (2) a DNA-dependent RNA polymerase II (pol II) promoter (CaMV 35S promoter) to drive the expression of Cas9 protein; (3) a cloning site located between the Pol III promoter and gRNA scaffold (FIG. 15C), which is used to insert a 20 by DNA sequence encoding the gRNA spacer for producing an engineered gRNA. In addition, a binary vector suitable for the Agrobacterium-mediated transformation was also constructed by inserting the same gRNA scaffold and Cas9 cassettes as those of pStGE3 into the T-DNA region in the pCAMBIA 1300 vector (see pStGEB3 in FIG. 15B).

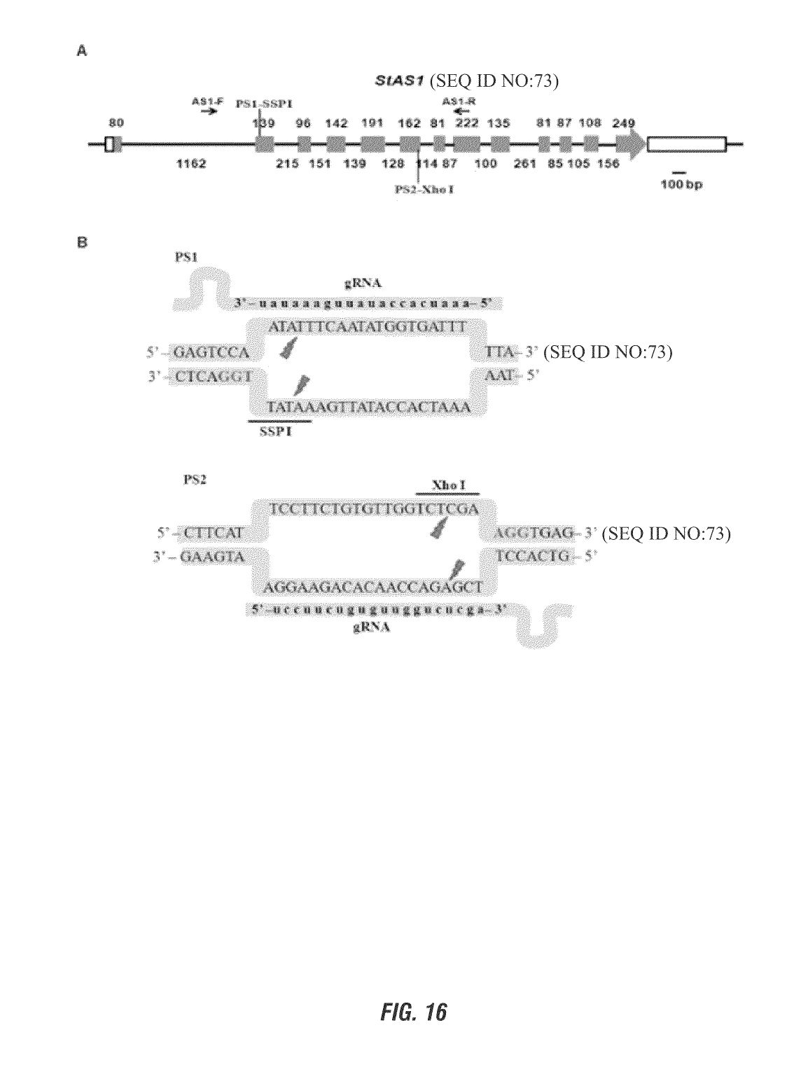

To demonstrate the CRISPR/Cas9 mediated genome editing in potato, the StAS1 gene which encodes an asparagine synthetase was chosen for targeted gene mutation. StAS1 was previously identified and characterized to regulate the accumulation of acrylamide in potato products such as French fries and potato chips. Therefore, a successful targeted mutation of StAS1 will significantly decrease the asparagine content in potato, leading to a reduction of acrylamide present in the processed potato products. Two guide RNA (gRNA) spacer sequences were designed based on the corresponding target sites in the StAS1 gene (PS1 and PS2, see FIG. 16). The Ps1-gRNA spacer (20 nt) was designed to pair with the template strand of StAS1, and contains a SspI restriction site, which will be destroyed if Cas9/gRNA editing works as predicted. The Ps2-gRNA spacer (20 nt) was predicted to pair with the coding strand of StAS1 containing a XhoI restriction site. Subsequently, PS1 and PS2 constructs were made by inserting the synthetic DNA oligonucleotides which encode the gRNA spacers into the pStGE3 vector.

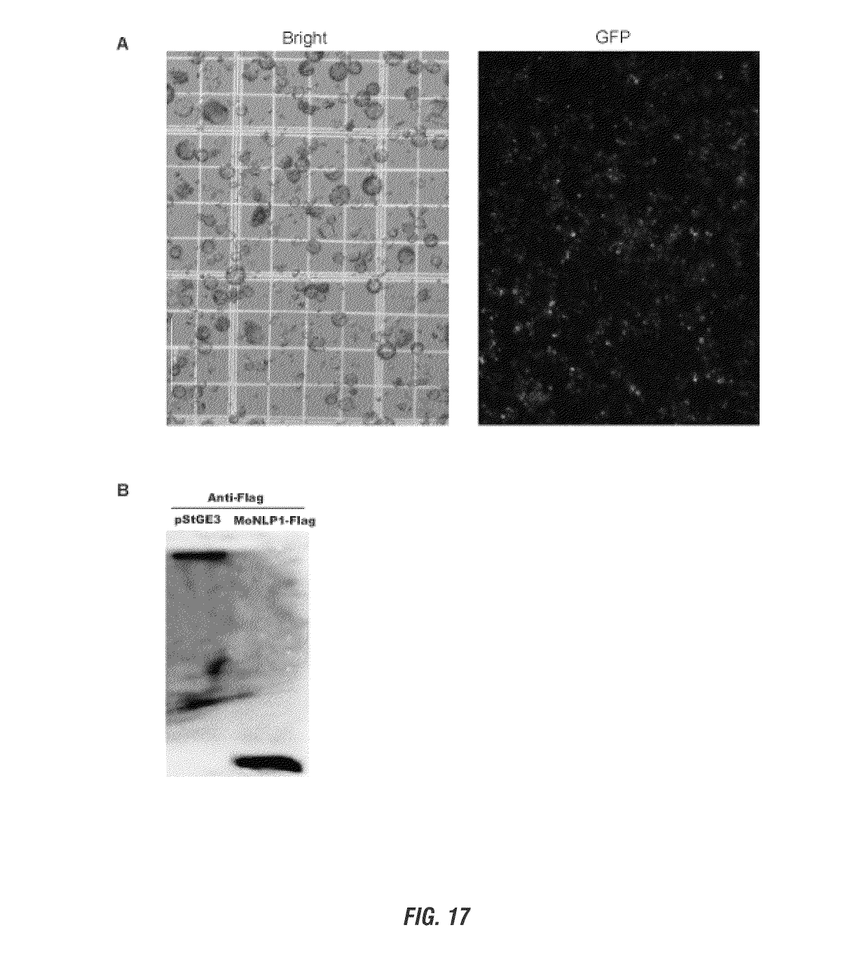

Protoplast transient expression system was used to test the PS1 and PS2 genome editing constructs. A simple and efficient procedure for the isolation and regeneration of protoplasts from tube potatoes was established previously, and a PEG-mediated transient transformation method has also been developed. Successful isolation and transfection of potato protoplasts was demonstrated using a plasmid construct carrying the green fluorescence protein (GFP) gene. Fluorescence microscopic analysis revealed the GFP expression in approximately 70% of the protoplasts at 24 hours after transformation (FIG. 17A). Following the transformation of empty pStGE3 vector and the pStGE3-PS1/2 gRNA constructs into potato protoplasts, the Cas9 nuclease was successfully expressed as shown by the immunoblot analysis (FIG. 17B).

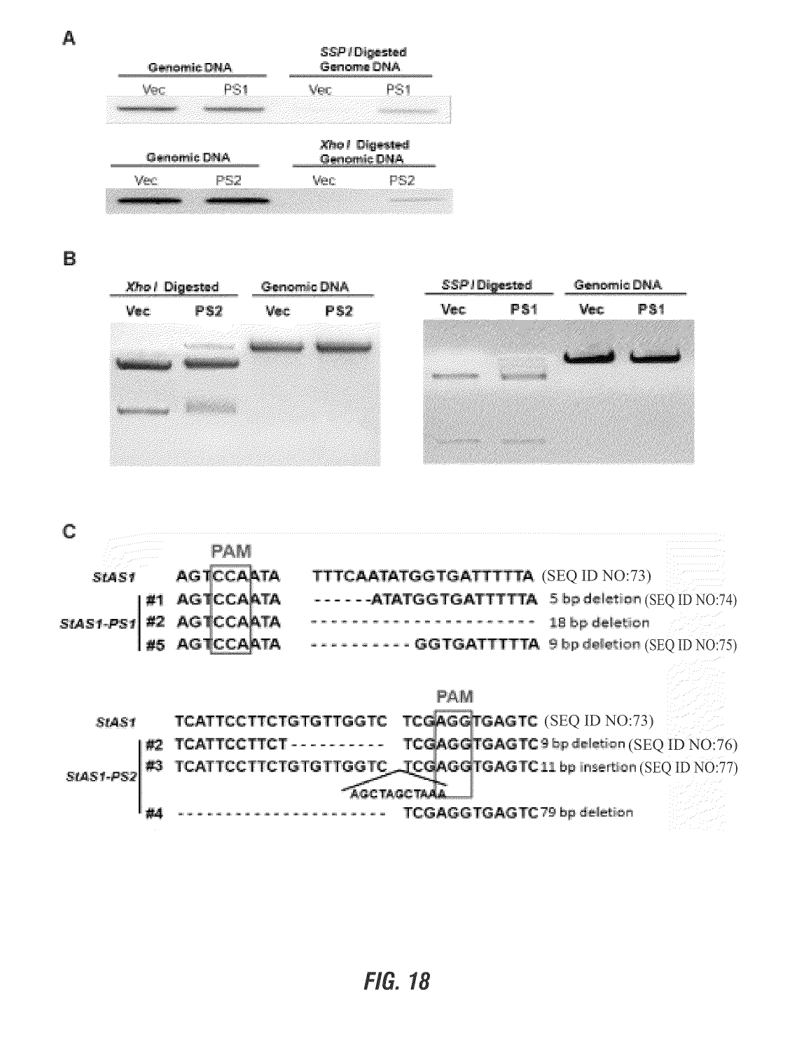

To detect the gRNA-guided genomic editing in protoplasts, potato genomic DNA was extracted from the transfected protoplasts at 24 hours after transformation. The extracted DNA was analyzed by RE-PCR as described in Example I, above. Before amplifying the StAS1 fragment, the genomic DNA was first digested by restriction enzyme to deplete wildtype StAS1. As a result, amplified StAS1 from the RE treated genomic DNA would enrich with targeted mutations that destroyed the restriction sites. Without restriction enzyme digestion, the yield of StAS1 PCR product (2.8 kb) was comparable between vector control and pStGE3-PS1 or PS2 transfected samples (FIG. 18A). However, after Ssp I or Xho I digestion, the 2.8 kb band was only detected in the DNAs extracted from protoplasts transformed with pStGE3-PS1 or pStGE3-PS2 constructs, but not detected in that from the vector control (FIG. 18A). Two additional replicates showed similar results with the same vectors (data not shown). In order to confirm this observation, we also applied PCR-RE (PCR-restriction enzyme digestion) assay to demonstrate targeted mutation of the StAS1 gene in potato protoplasts. The PCR products were first amplified from genomic DNAs using a pair of specific primers (StAS1-F and StAS1-R), and then digested with SspI or XhoI. Without restriction enzyme digestion, the expected PCR fragment (2.7 kb) was revealed by agarose gel electrophoresis. However, a 700 by fragment and a 2.1 kb fragment were found with the SspI digested PCR product from the pStGE3 vector transformed protoplasts. By contrast, a 2.8 kb DNA fragment was found with the SspI digested PCR products from the the pStGE3-PS1 transformed protoplasts (FIG. 18B). For pStGE3-PS2 construct, a similar result was obtained with a 2.8 kb fragment from the pStGE3-PS2 samples compared to 800 by and 2 kb digested fragments from the pStGE3 vector transformed sample. The mutation efficiency was also estimated based on PCR-RE assay results (FIG. 18B) by calculating the percentage of mutated fraction which resistant to SspI or Xho I digestion. In pStGE3-PS1 samples, the mutation rate was estimated to be 3.6%, and pStGE3-PS2 samples showed a similar mutation rate about 4.6%. These data suggest that targeted mutations which destroyed the Ssp I and Xho I sites in StAS1 were successfully introduced in potato genome by engineered Cas9-gRNA.

The PCR products from pStGE3-PS1/PS2 samples were purified using gel purification kit (Qiagen) and cloned into pGEM-T vector for sequencing. A total of ten clones were sequenced. These sequencing data further confirmed that targeted mutations were introduced at the predicted Cas9 cleavage site, which is 3 by upstream of PAM sequence (FIG. 18C). Further analysis revealed that the mutations were resulted from either nucleotide deletions or insertion (FIG. 18C). These results demonstrate that the engineered CRISPR/Cas9 system can precisely create double-strand breaks at specific sites of the potato genome, leading to targeted gene mutations by the NHEJ DNA repairing machinery.

### Plant Materials

Four to six week old potato plants were grown in a greenhouse (23-25° C.). Solanum tuberosum DM1-3 516 R44 (referred to as DM), the sequenced cultivar from doubled monoploid clone derived classical tissue culture, was provided by Dr. Veilleux at USDA and Virginia Tech.

### Construction of RNA-Guided Genome Editing Vectors

To construct pStGE3 vector, snoRNA U3 promoters were amplified from Arabidopsis cultivar Columbia genomic DNA using primer pairs gRNA-BamHI-F/BsaI-AtU3b-R. The DNA sequence encoding the gRNA scaffold was amplified from pX330a vector (Cong et al., 2013) using a pair of primers (Bsa-gRNA-F and rRNA-HindIII-R). The PCR product of U3 promoter was fused with the DNA fragment encoding gRNA scaffold by overlapping PCR. The U3 promoter-gRNA fragment was then cloned into the BamH/HindIII double digested site of pUC19-BsaI vector to produce pUC19-AtU3-gRNA. pUC19-BsaI was derived from pUC19 (Nakagawa et al., 2007) by removing one Bsa I sites in ampicillin resistance gene using site-directed mutagenesis (Agilent Technologies). The Cas9 gene fragment was amplified from pX330a with a pair of primers (Cas9-KpnI-F and Cas9-KpnI-R) using High-Fidelity phusion polymerase and then inserted into KpnI digested pUC19-AtU3-gRNA vector, resulting in the pStGE3 vector (FIG. 15A).

### Gene Constructs for Targeted Gene Mutation

DNA sequences encoding gRNAs were designed to target two specific sites in the exons of StAS1 (FIG. 16A). For each target site, a pair of DNA oligonucleotides with appropriate cloning linkers were synthesized (IDT, Inc). Each pair of oligonucleotides were phosphorylated, annealed, and then ligated into BsaI digested pStGE3 vectors. After transformation into E. coli DH5-alpha, the resulting constructs were purified with QIAGEN Plasmid Midi kit (Qiagen) for subsequent use in potato protoplast transformation.

### Potato Protoplast Preparation and Transformation

Potato protoplasts were prepared from 4-6 week-old potato leaves of DM cultivar (Diploid Solanum tuberosum). Potato leaves were first incubated in conditional medium containing 1× MS, 100 mg/L Casein hydrolysate, 3 mM MES pH 5.7, 0.35 M Mannitol, 2 mg/L NAA and 1 mg/L BA. Then the protoplasts were isolated by digesting these potato leaves in Digestion Solution (1× MS, 3 mM MES pH5.7, 0.3 M Mannitol, 1 mM CaCl2, 5 mM beta-mercaptoethanol, 0.2% BSA, 1% Cellulase R10 [Yakult Pharmaceutical, Japan], and 0.375% Macerozume R10 [Yakult Pharmaceutical, Japan]) for 3.5 hours. After filtering through Nylon mesh (35 um), the protoplasts were washed by W5 solution (2 mM MES pH5.7, 154 mM NaCl, 5 mM KCl, 125 mM CaCl2) at room temperature (25° C.) 3-5 times and then collected and incubated in W5 solution for 30 minutes. The W5 solution was then removed by centrifugation at 300×g for 3 min, and potato protoplasts were resuspended in MMG solution (4 mM MES, 0.6 M Mannitol, 15 mM MgCl2) to a final concentration of 5.0×106/ml. For transformation, 10 ul of plasmids (5-10 ug) was gently mixed with 100 ul of protoplasts and 110 ul of PEG-CaCl2 solution (0.6 M Mannitol, 100 mM CaCl2 and 40% PEG4000), and then incubated at room temperature for 20 min. Transformation was stopped by adding 2× volume of W5 solution. Transformed protoplasts were then collected by centrifugation and resuspended in W5 solution. The transformed protoplasts were maintained in 24-well culture plates. After 24-48 hours of incubation in W5 solution, protoplasts were collected by centrifugation at 300×g for 2 min and frozen in −80° C. for further analysis.

### Western Blotting and Immunodetection

To extract total proteins, 100 ul of Lysis Buffer (25 mM Tris-HCl pH7.5, 150 mM NaCl, 2% Triton X-100, 10% glycerol, 5 ug/mL protease inhibitor cocktail [Sigma-Aldrich]) was added to 2×106 potato protoplasts. The cell debris was removed by centrifugation at 12000 rpm for 15 min. Ten microliter of protein extract was separated by 10% SDS-PAGE and transferred to PVDF membrane. The Cas9-FLAG fusion protein was detected with the anti-FLAG antibody (Sigma-Aldrich).

### Genomic DNA Extraction

Genomic DNA was extracted from potato protoplasts by adding 150 ul of extraction buffer (200 mM Tris-HCl PH 7.5, 250 mM NaCl, 25 mM EDTA, 0.5% SDS, 10 mg/L Rnase I) and shaking the mixture for 1 min. After centrifugation at 12000 rpm for 5 min, the supernatant was transferred to a new tube and mixed with 150 isopropyl alcohol. Following incubation on ice for 20 min, genomic DNA was precipitated by centrifugation at 12000 rpm for 15 min at 4° C. The DNA pellet was washed with 0.5 ml of 70% ethanol and air dried. The genomic DNA was then dissolved in 80 ul of H2O and its concentration was determined by spectrophotometer.

### Restriction Enzyme Digestion Suppressed PCR

To detect mutation at desired restriction enzyme sites, 500 ng of genomic DNA was digested with Ssp I (Vector and StAS1-PS1) or Xho I (Vector and StAS1-PS2) at 37° C. for 2-4 hours. The DNA fragments containing the gRNA-Cas9 target sites were then amplified by PCR from the digested and un-digested genomic DNAs. The PCR products were analyze by electrophoresis in 1% agrose gel (FIG. 18A). To identify targeted gene mutation, purified PCR products from RE digested template were cloned to pGEM-T easy vector by TA cloning (Promega), and resulting colonies were used for plasmid extraction and DNA sequencing. To determine mutation rate on PS1-and PS2-gRNA target sites, we also performed PCR-RE digestion experiment. DNA extracted from StAS1-PS1 and StAS1-PS2 transfected protoplasts were amplified using primers StAS1-F and StAS1-R. The amplicon was then digested with SspI or XhoI. Mutated, un-digestable DNA fragment were detected by agrose gel electrophoresis (FIG. 18B).

### DNA Sequencing

After the initial PCR detection of targeted mutation, the cloned fragments in pGEM-T were sequenced by the conventional Sanger sequencing (see FIG. 18C).

### Accession Numbers

Sequence data from this example can be found in the EMBL/GenBank data libraries under accession number: StAS1 (XM_{—}006343993.1), pUC19 (M77789.2).

TABLE 3
Oligonucleotides used to generate
pStGE3 and pStGEB3 vectors and the StAS1 targeting construct.
Oligonucleotides for constructing plasmid vectors

| | | |
|---|---|---|
| Arabidopsis | gRNA-BamHI-F | TAGGATCCCAGCCTGTGATGGATAACTG (SEQ |
| U3 promoter | | ID NO: 36) |
| | BsaI-AtU3B-R | CGAGACCTCGGTCTCTGACCAATGTTGCTCCC |
| | | TCAGT (SEQ ID NO: 37) |
| gRNA scaffold | BsaI-gRNA-F | AGAGACCGAGGTCTCGGTTTTAGAGCTAGAA |
| | | ATA (SEQ ID NO: 38) |
| | gRNA-HindIII-R | TCAAGCTTCGCGCTAAAAACGGACTAG (SEQ |
| | | ID NO: 39) |
| 35S:Cas9 | Cas9-KpnI-F | TCGGTACCCAGGTCCCCAGATTAGCCTT (SEQ |
| elements | | ID NO: 40) |
| | Cas9-KpnI-R | TCGGTACCGACGTTGTAAAACGACGGCC (SEQ |
| | | ID NO: 41) |

Oligonucleotides for generating DNA sequences
encoding gRNAs for targeting the StAS1 gene

| | | |
|---|---|---|
| StAS1-PS1 | StASN1 PS1-F | GGTCATATTTCAATATGGTGATTT (SEQ ID NO: 42) |
| | StASN1 PS1-R | AAACAAATCACCATATTGAAATAT (SEQ ID |
| | | NO: 43) |
| StAS1-PS2 | StASN1 PS2-F | GGTCTTCCTTCTGTGTTGGTCTCG (SEQ ID |
| | | NO: 44) |
| | StASN1 PS2-R | AAACCGAGACCAACACAGAAGGAA (SEQ ID |
| | | NO: 45) |
| Primer for | StASN1-F | TCAGTTGAACCTGCGGAATT (SEQ ID NO: 46) |
| StAS1 | StASN1-R | TCGATACTCATGGCAACATC (SEQ ID NO: 47) |
| genomic DNA | | |

### Example III

### Targeted Mutation of AtPDS3 in Arabidopsis via the Agrobacterium tumefaciens-Mediated Transformation

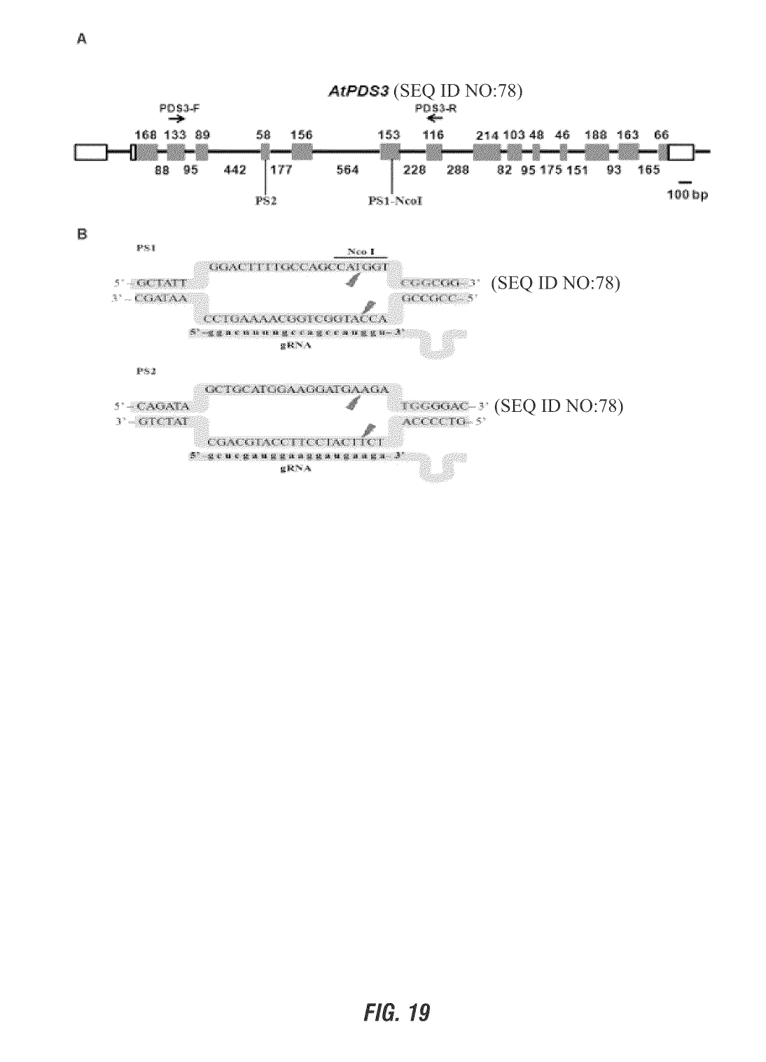

To test if the gRNA-Cas9 system works in the Agrobacterium-mediated plant transformation, Two gRNAs were designed to target two distinct sites in the coding region of AtPDS3 (Accession number: NM_{—}202816.2) which encodes the Arabidopsis phytoene dehydrogenase (FIG. 19). Plants defective in AtPDS3 display leaf bleaching phenotype, which makes it easy to examine gene knock-out efficiency. Two DNA sequences (Table 4) encoding the gRNAs were synthesized and cloned into pRGEB3 and pStGEB3, respectively.

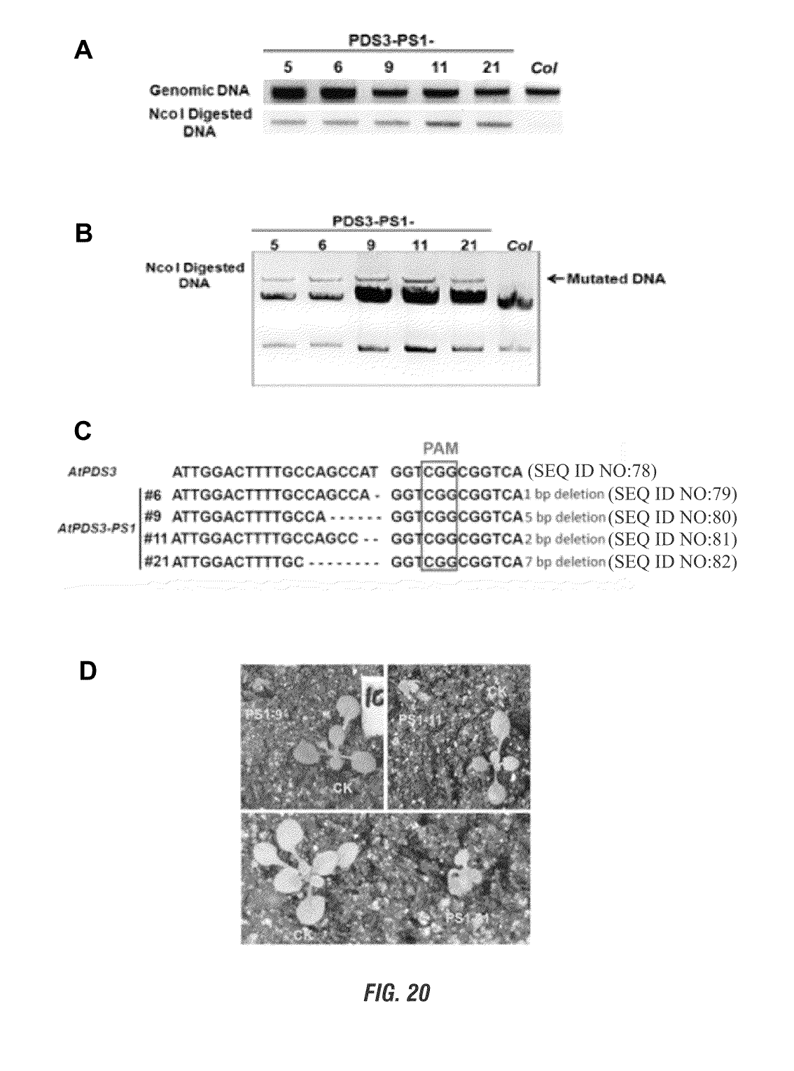

Two sets of RGE vectors were used for targeted mutagenesis of AtPDS3 in Arabidopsis using the Agrobacterium tumafaciens-mediated floral dip method. One contains the 35S promoter-driven Cas9 and rice U3 promoter-driven gRNA in pRGEB3, while another contains the 35S promoter-driven Cas9 and Arabidopsis U3 promoter-driven gRNA in pStGEB3. Following the Agrobacterium-mediated transformation with the pRGEB3 construct, 38 transgenic Arabidopsis lines were analyzed and found to express Cas9 protein. However, targeted mutation of AtPDS3 was not detected in any of these transgenic lines using the RE-PCR method. By contrast, 24 transgenic Arabidopsis lines were analyzed after the Agrobacterium-mediated transformation with the pStGEB3 construct. Based on the RE-PCR and DNA sequencing analysis, targeted mutation of AtPDS3 was detected in at least 5 out of 24 transgenic lines (FIG. 20). It is likely that the absence of targeted mutation with pRGEB3 might result from the low expression of rice U3 promoter-driven gRNA in Arabidopsis or dicot plants. Therefore, Arabidopsis U3 promoter is more efficient to express gRNA for genome editing in dicots, whereas rice U3 promoter is more efficient to express gRNA for genome editing in monocots and cereal crops.

TABLE 4
Oligonucleotides used to make the gRNA-encoding
DNA molecules targeting the AtPDS3 gene.

| | |
|---|---|
| PDS3-PS1-F | 5′-GGTTGCAAAGTACCTGGCTGATGC-3′ |
| | (SEQ ID NO: 48) |
| PDS3-PS1-R | 5′-AAAC GCATCAGCCAGGTACTTTGC-3′ |
| | (SEQ ID NO: 49) |
| PDS3-PS2-F | 5′-GGTT ATCAATGATCGGTTGCAGTGGA-3′ |
| | (SEQ ID NO: 50) |
| PDS3-PS2-R | 5′-AAAC TCCACTGCAACCGATCATTGAT-3′ |
| | (SEQ ID NO: 51) |

### Example IV

### Genome-Wide Prediction of Highly Specific Guide RNA Spacers for CRISPR—Cas9-Mediated Genome Editing in Model Plants and Major Crops

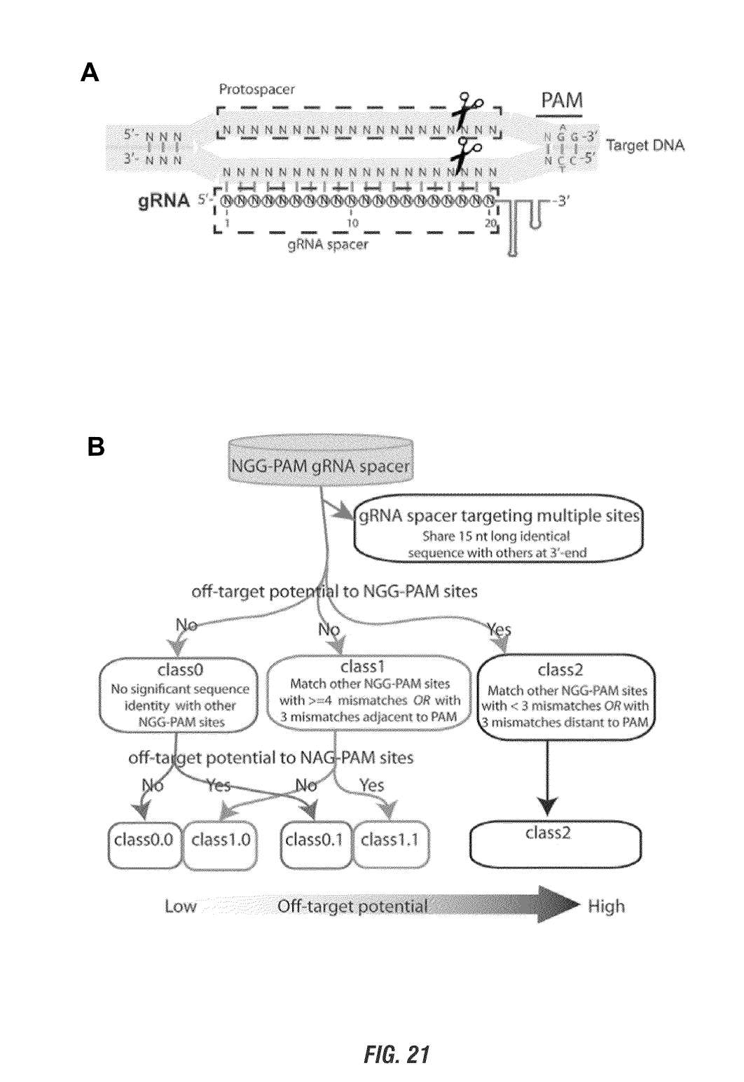

RNA-guided genome editing (RGE) using the Streptococcus pyogenes CRISPR—Cas9 system (Jinek et al., 2012; Cong et al., 2013; Mali et al., 2013b) is emerging as a simple and highly efficient tool for genome editing in many organisms. The Cas9 nuclease can be programmed by dual or single guide RNA (gRNA) to cut target DNA at specific sites, thereby introducing precise mutations by error-prone non-homologous end-joining repairing or by incorporating foreign DNAs via homologous recombination between target site and donor DNA. The gRNA—Cas9 complex recognizes targets based on the complementarity between one strand of targeted DNA (referred as protospacer) and the 5′-end leading sequence of gRNA (referred to as gRNA spacer) that is approximately 20 base pairs (bp) long (FIG. 21A). Besides gRNA—DNA pairing, a protospacer-adjacent motif (PAM) following the paired region in the DNA is also required for Cas9 cleavage. Recent studies reveal that Cas9 could cut the PAM-containing DNA sites that imperfectly match gRNA spacer sequences, resulting in genome editing at undesired positions. This off-target editing of engineered gRNA—Cas9 has been extensively examined recently (Hsu et al., 2013; Mali et al., 2013a). Thus, gRNA—Cas9 specificity becomes a major concern for RGE application, and it is very important to evaluate the potential constraint of Cas9 specificity and develop straightforward bioinformatics tools to facilitate the design of highly specific gRNAs to minimize off-target effects.

Nucleotide mismatch between a gRNA spacer sequence and a PAM-containing genomic sequence was shown to significantly reduce the Cas9 affinity at the target site in vitro or in animal cells (Hsu et al., 2013; Mali et al., 2013a; Pattanayak et al., 2013). Cas9 generally tolerates no more than three mismatches in the gRNA—DNA paired region and the presence of mismatches adjacent to PAM would greatly reduce Cas9 affinity to the site imperfectly matching the gRNA. Thus, the off-target risk of a designed gRNA could be assessed by similarity searching against whole-genome sequence in silico; and, vice versa, genome-wide sequence analysis could be used to predict gRNA spacer with high specificity for RGE in designated specie. For plants, especially crops whose genome sizes range from ˜1×10⁸ to 2×10⁹ by with different levels of sequence complexity and duplication, genome-wide prediction of specific gRNAs would help evaluate the potential constraint for Cas9 off-target effects and greatly facilitate the application of the RGE technology in plant functional genomics and genetic improvement of agricultural crops. To this end, the Inventors analyzed the assembled nuclear genome sequences of eight representative plant species (Table 5), including Arabidopsis thaliana, Medicago truncatula, Glycine max (soybean), Solanum lycopersicum (tomato), Brachypodium distachyon, Oryza sativa (rice), Sorghum bicolor, and Zea mays (maize) to predict specific gRNA spacers which are expected to have little or no off-target risk in RGE.

TABLE 5
Data sources of the analyzed plant genomes.

| | | | | Genome |
|---|---|---|---|---|
| | | GenBank Assembly | Release | Annotation |
| Species | Group | ID | version | Source |
| Arabidopsis thaliana | dicot | GCA_000001735.1 | TAIR10 | TAIR |
| Medicago truncatula | dicot | GCA_000219495.1 | Mt3.5V4 | MIPS |
| Solanum lycopersicum | dicot | GCA_000188115.1 | SL2.40 | MIPS |
| Glycine max | dicot | GCA_000004515.1 | v1.1 | Phytozome |
| Brachypodium distachyon | monocot | GCA_000005505.1 | v1.2 | MIPS |
| Oryza sativa | monocot | GCA_000005425.2 | RGAP release 7 | RGAP |
| Sorghum bicolor | monocot | GCA_000003195.1 | Sorghum1.4 | MIPS |
| Zea mays | monocot | GCA_000005005.4 | B73 RefGen_v2: | maizeGDB |
| | | | Release 5b.59 | |

TAIR, The Arabidopsis Information Resource: http://www.arabidopsis.org/index.jsp
RGAP, Rice Genome Annotation Project: http://rice.plantbiology.msu.edu
Phytozome,: http://www.phytozome.net/
MIPS PlantsDB: http://mips.helmholtz-muenchen.de/plant/genomes.jsp
MaizeGDB: http://maizegdb.org/

The genome sizes of the selected plants span the range of 120-2065 Mb (Table 6) and represent most of land plants. Assembled chromosome sequences were downloaded from NCBI Genebank except Arabidopsis thaliana and Oryza sativa whose genome sequences were downloaded from TAIR and the RGAP website (Table 5), respectively. Non-nuclear genome sequences (plastid and mitochondrion genomes) and unplaced sequences were excluded in the analysis. The sources of sequence and annotation data are shown in Table 5.

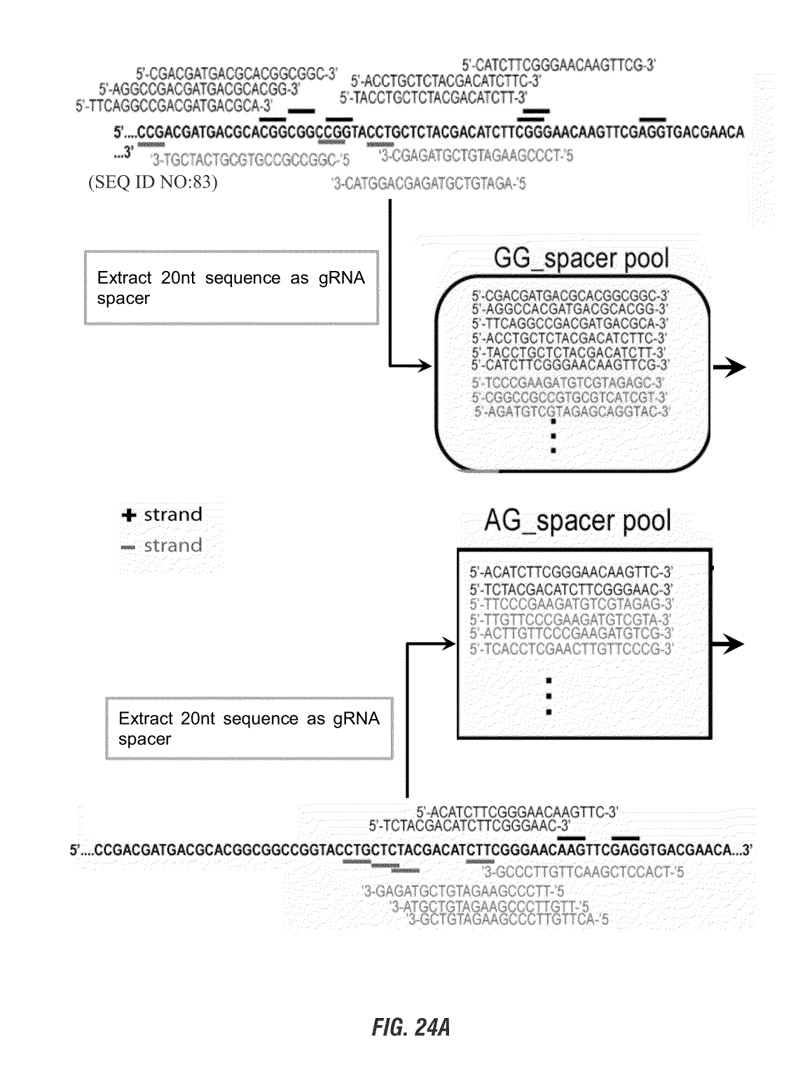

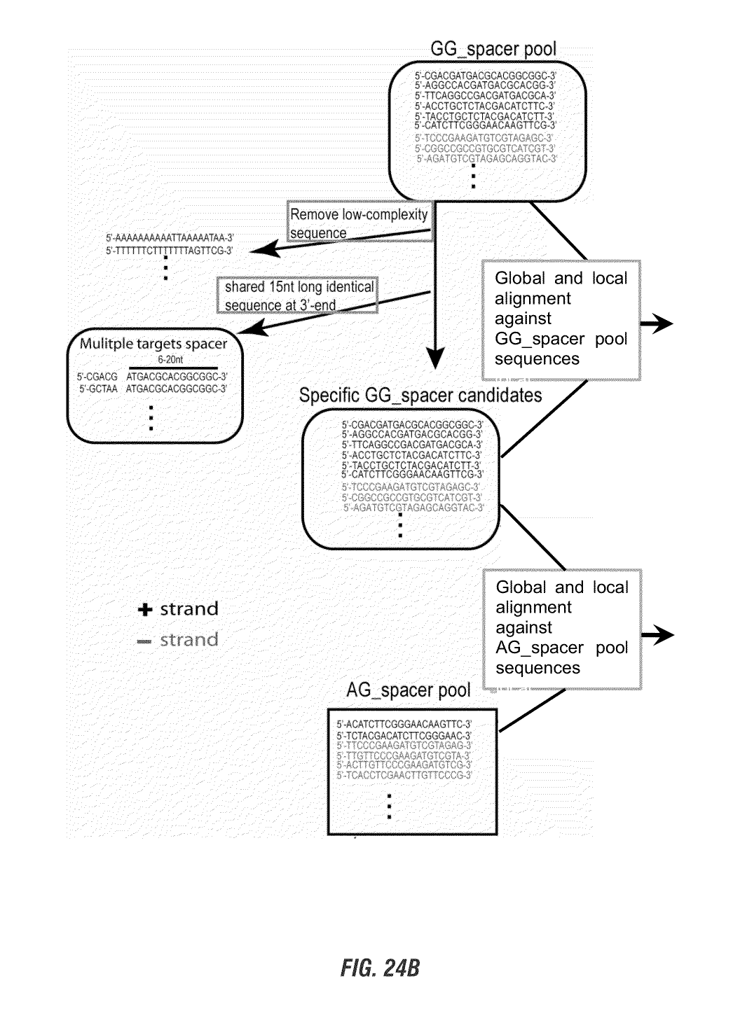

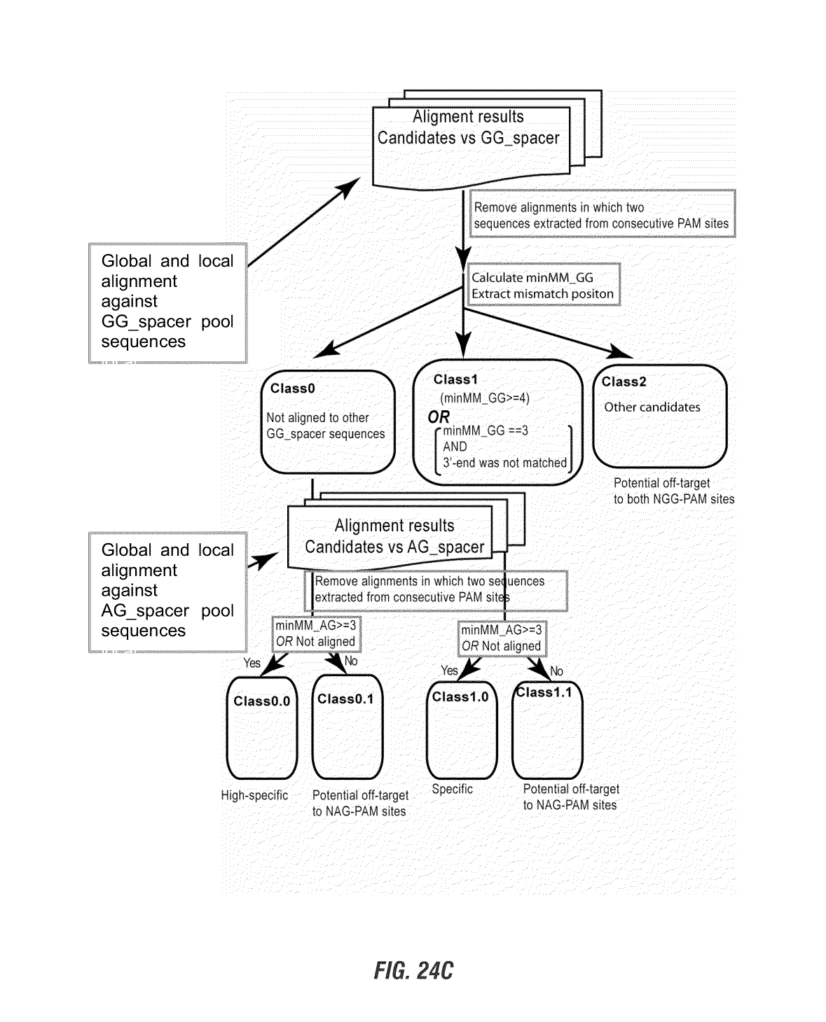

The choice of gRNA spacer sequences is limited to locations with PAMs in the genome. The gRNA—Cas9 complex recognizes two PAMs, 5′-NGG-3′ and 5′-NAG-3′, but shows much less affinity and less tolerance of mismatches at the NAG—PAM site (Hsu et al., 2013). Thus, only specific gRNA spacers targeting NGG—PAM sites were predicted. Potential gRNA spacer sequences (20 nt long) were extracted from the genomic sequences before NGG—PAM (GG-spacer). The 20-nt sequences before NAG—PAM (AG-spacer) were also extracted, but only used off-target assessment. The off-target risk of a gRNA spacer is dependent on its similarity to all GG-spacers and AG-spacers. After the pair-wise sequence comparison, two steps were taken to classify these GG-spacer sequences according to their off-target potential (FIG. 21B; see details in Methods, FIG. 24, and Table 6). First, each GG-spacer was sorted to Class0 (no significant sequence similarity with other GG-spacers), Class1 (four or more mismatches, or three mismatches adjacent to PAM in all GG-spacer alignments), or Class2 (fewer than three mismatches, or three mismatches distant to PAM in all GG-spacer alignments). A Class2 candidate is considered to have off-target possibilities because it shares significant sequence identity with other GG-spacers and contains fewer mismatches. Second, GG-spacers from Class0 and Class1 were further classified to subclasses after comparing with all AG-spacers. Class0.0 and Class1.0 spacers are expected to be highly specific whereas Class0.1 and Class1.1 may cause off-target effects on other NAG—PAM sites. A GG-spacer may have off-target effects on other NAG-sites if it matches other AG-spacers with fewer than three mutations. These criteria were selected based on the recent reports regarding the gRNA specificity and off-target analyses in animals (Hsu et al., 2013; Mali et al., 2013a; Pattanayak et al., 2013) and observations in plants (Li et al., 2013; Nekrasov et al., 2013; Shan et al., 2013; Xie and Yang, 2013). As a result, Class0.0 and Class1.0 gRNA spacers are expected to provide high specificity in the CRISPR—Cas9-mediated genome editing, with class0.0 gRNA spacers being the most specific.

TABLE 6
Summary of specific gRNA spacer prediction.

| | Species | | | | | | | |
|---|---|---|---|---|---|---|---|---|
| | At | Mt | Sl | Gm | Bd | Os | Sb | Zm |
| Genome size | 119.67 | 314.48 | 781.5 | 973.49 | 272.06 | 382.78 | 739.15 | 2065.7 |
| (×10⁶ bp) | | | | | | | | |
| Chromosome | 5 | 8 | 12 | 20 | 5 | 12 | 10 | 10 |
| number | | | | | | | | |
| NGG-PAM | 8045909 | 15624099 | 49470191 | 68255111 | 30578740 | 38923015 | 64728281 | 246261552 |
| NAG-PAM | 14137505 | 26050018 | 80831959 | 104930271 | 33033062 | 43923904 | 79413270 | 262207278 |
| Candidate | 5746294 | 7472598 | 21087048 | 21495656 | 17567744 | 18567257 | 22061504 | 32974088 |
| gRNA spacers | | | | | | | | |
| Class0 gRNA | 44267 | 118727 | 31396 | 33834 | 14095 | 12087 | 5185 | 83 |
| spacers | | | | | | | | |
| Class0.0 | 43682 | 115198 | 30211 | 31641 | 13743 | 11677 | 4982 | 78 |
| Class0.1 | 585 | 3529 | 1185 | 2193 | 352 | 410 | 203 | 5 |
| Class1 gRNA | 4406732 | 5108299 | 9634226 | 10010742 | 12072172 | 12078614 | 13486412 | 13150408 |
| spacers | | | | | | | | |
| Class1.0 | 4083627 | 4077138 | 6549562 | 6520868 | 10628745 | 10068167 | 11041168 | 10180017 |
| Class1.1 | 323105 | 1031161 | 3084664 | 3489874 | 1443427 | 2010447 | 2445244 | 2970391 |
| Specific gRNA | 4127309 | 4192336 | 6579773 | 6552509 | 10642488 | 10079844 | 11046150 | 10180095 |
| spacers | | | | | | | | |
| (Class0.0 and 1.0) | | | | | | | | |
| Class2 gRNA | 1295295 | 2245572 | 11421426 | 11451080 | 5481477 | 6476556 | 8569907 | 19823597 |
| spacers | | | | | | | | |

At, Arabidopsis thaliana;
Mt, Medicago truncatula;
Sl, Solanum lycopersicum;
Gm, Glycine max;
Bd, Brachypodium distachyon;
Os, Oryza sativa;
Sb, Sorghum bicolor;
Zm, Zea mays.

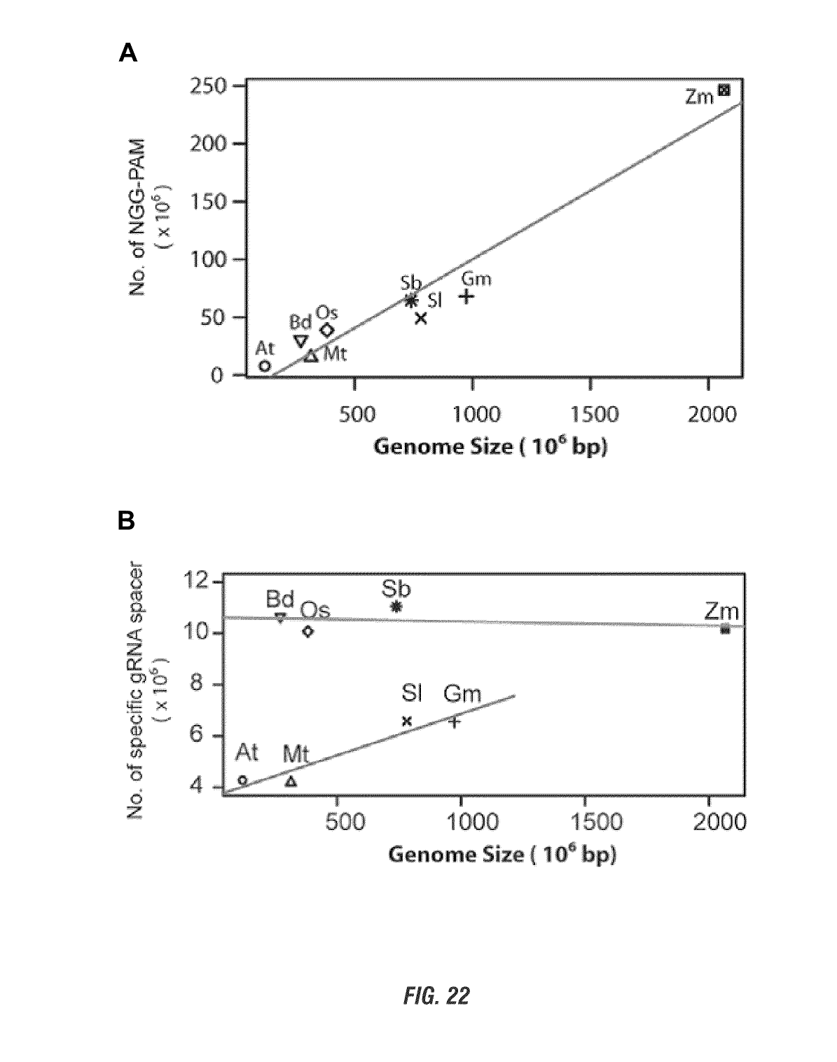

Among these eight plant species, 5-12 NGG—PAMs were identified every 100 by in chromosomes (Table 7), and the total number of NGG—PAMs is positively correlated to genome size (correlation coefficient R=0.97, FIG. 22A). The total number of specific gRNA spacers (Class0.0 and 1.0) ranges from 4 to 11 million, and more specific gRNAs were predicted in monocots (Brachypodium, rice, Sorghum, and maize) than in eudicots (Arabidopsis, Medicago, tomato, and soybean) despite their genome size. The number of specific gRNA spacers is positively correlated to genome size (R=0.95) in four eudicot species (FIG. 22B). In four monocot species, however, the number of specific gRNA spacers is not proportional to the genome size (R=−0.30, FIG. 22B), nor to the total transcript number (R=−0.67) or the NGG—PAM number (R=−0.37). Comparable numbers of specific gRNA spacers (10-11×10⁶) were found in four monocot species despite the significant difference (two to eight-fold) in their genome sizes (FIG. 22B and Table 6). Although the 20-nt-long gRNA spacer sequences have more chance to be aligned with other PAM sites with fewer mismatches in bigger genomes, the number of specific gRNA spacers also depends on the genome sequence content.

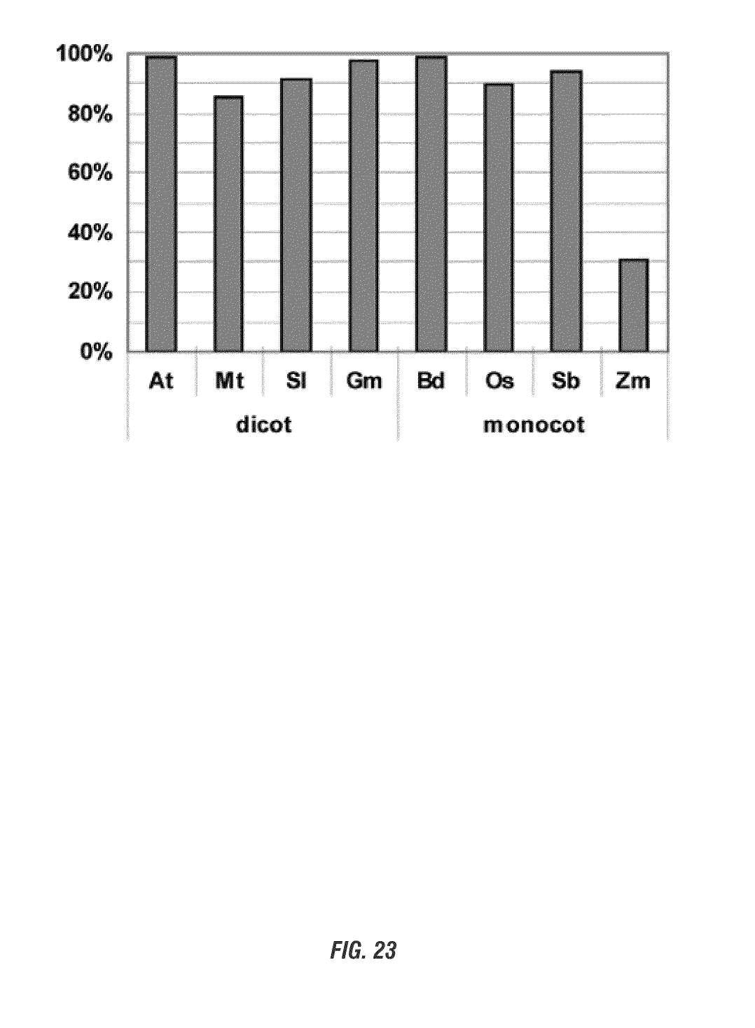

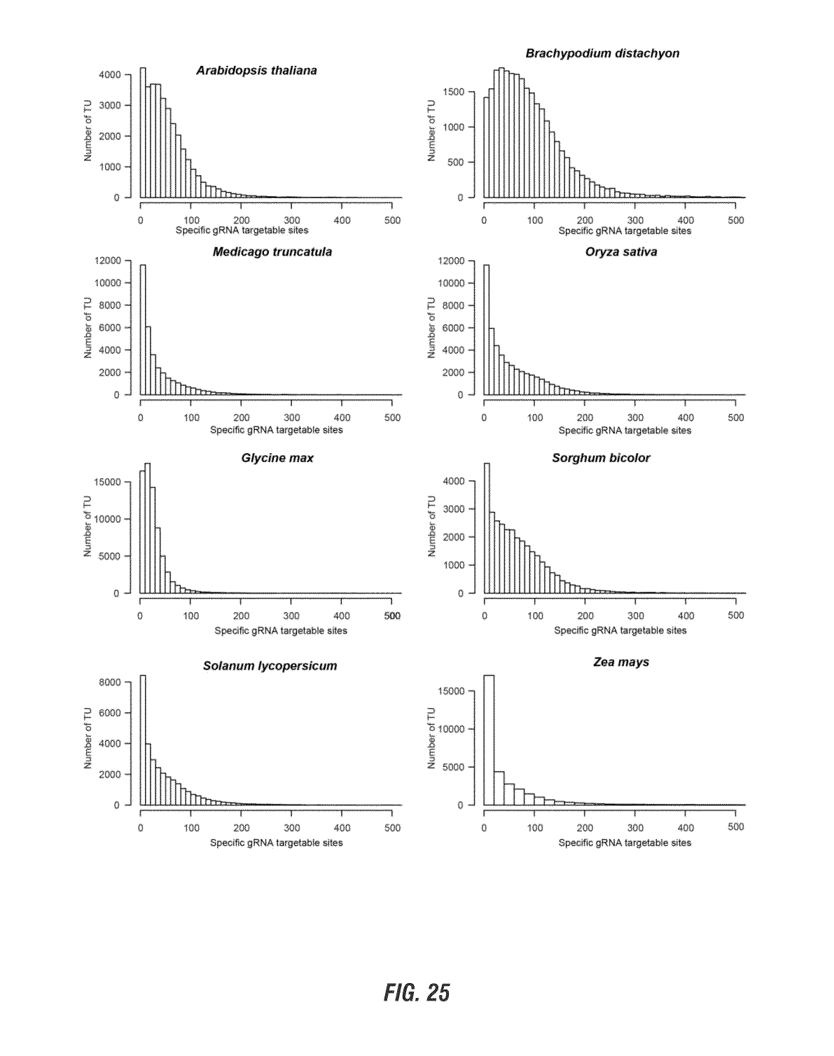

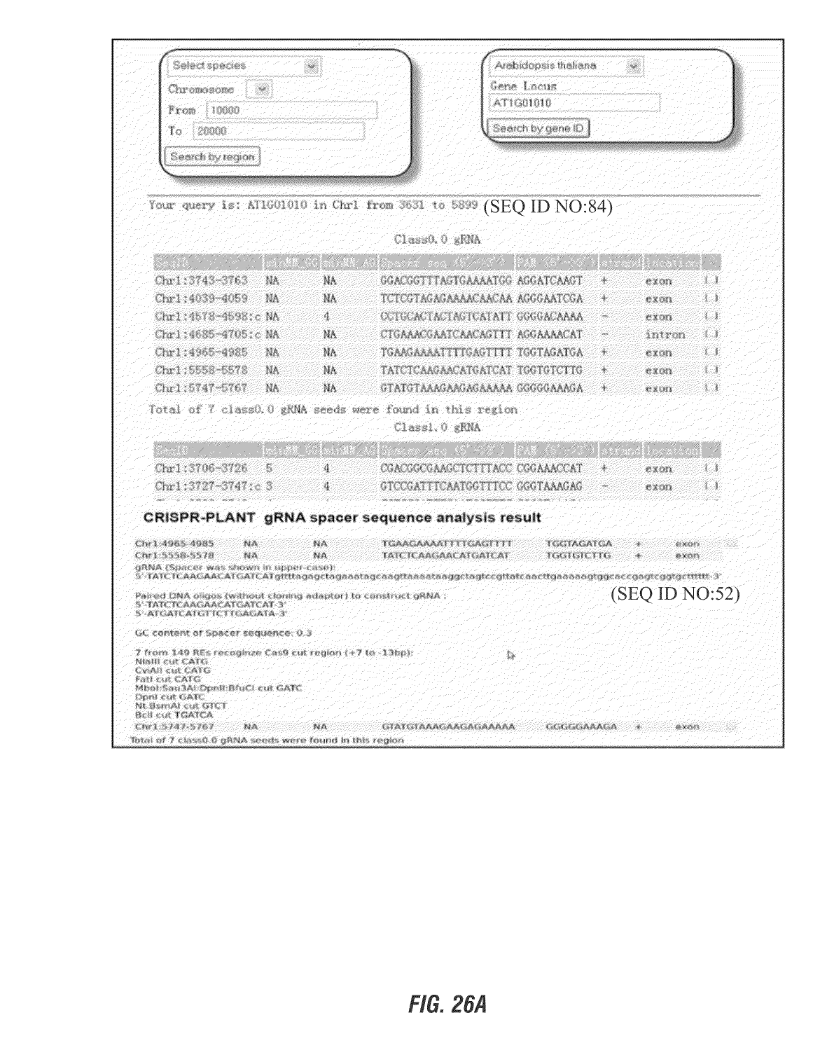

The proportion of annotated genes that could be targeted by specific gRNAs designed from Class0.0 and Class1.0 spacer sequences was calculated. Based on the current genome annotation for seven of the eight plant species, specific gRNAs could be designed to target 85.4%-98.9% of annotated transcript units (TU), and 83.4%-98.6% of TUs could be targeted in exons (FIG. 23 and Table 7). The exception, maize, has the largest genome and the largest number of annotated TUs among these eight species, but only 30% of maize TUs are targetable by the specific gRNA (Table 7). For the other seven plant species, 67.9%-96.0% of TUs have at least 10 NGG—PAM sites that could be targeted by specific gRNAs containing Class0.0 or Class1.0 spacers (FIG. 25). Thus, the off-target effect of CRISPR—Cas9 could be minimized and will not constrain genome editing in Arabidopsis, Medicago, tomato, soybean, rice, Sorghum, and Brachypodium.

TABLE 7
Summary of annotated transcript units (TUs) targetable by specific gRNA spacers.

| Species | | | | | | | |
|---|---|---|---|---|---|---|---|
| At | Mt | Sl | Gm | Bd | Os | Sb | Zm |

No. of TUs targetable by specific gRNA

| | | | | | | | | |
|---|---|---|---|---|---|---|---|---|
| Class0.0 | 15501 | 19128 | 8772 | 14460 | 4023 | 4330 | 1324 | 20 |
| | (47.0%) | (46.5%) | (25.3%) | (19.8%) | (15.2%) | (7.8%) | (3.9%) | (.%) |
| Class1.0 | 32042 | 35076 | 31653 | 71094 | 26213 | 50005 | 31935 | 33452 |
| | (97.1%) | (85.3%) | (91.1%) | (97.3%) | (98.8%) | (89.6%) | (93.9%) | (30.5%) |
| Class0.0 and | 32045 | 35113 | 31657 | 71097 | 26213 | 50008 | 31935 | 33452 |
| Class1.0 | (97.1%) | (85.4%) | (91.2%) | (97.3%) | (98.8%) | (89.6%) | (93.9%) | (30.5%) |

No. of TUs with specific gRNA targetable sites in exon

| | | | | | | | | |
|---|---|---|---|---|---|---|---|---|
| Class0.0 | 14717 | 16438 | 7043 | 11301 | 2377 | 2872 | 782 | 8 |
| | (44.6%) | (40.%) | (20.3%) | (15.5%) | (9.%) | (5.1%) | (2.3%) | (.%) |
| Class1.0 | 31123 | 34244 | 31088 | 70409 | 26138 | 48717 | 31510 | 32385 |
| | (94.3%) | (83.3%) | (89.5%) | (96.4%) | (98.6%) | (87.3%) | (92.6%) | (29.5%) |
| Class0.0 and | 31125 | 34286 | 31092 | 70412 | 26138 | 48720 | 31510 | 32385 |
| Class1.0 | (94.3%) | (83.4%) | (89.5%) | (96.4%) | (98.6%) | (87.3%) | (92.6%) | (29.5%) |

At, Arabidopsis thaliana;
Mt, Medicago truncatula;
Sl, Solanum lycopersicum;
Gm, Glycine max;
Bd, Brachypodium distachyon;
Os, Oryza sativa;
Sb, Sorghum bicolor;
Zm, Zea mays.

The inventors further examined the feasibility of specifically targeting the nucleotide-binding site leucine-rich repeat (NBS—LRR) genes, which comprise one of the largest plant gene families and evolve rapidly to mediate host resistance against pathogen infection. The number of predicted NBS—LRR genes varies from 112 to 502 in these eight species (Table 8). Specific gRNAs could be designed to target almost all NBS—LRR genes in Arabidopsis, soybean, rice, tomato, Brachypodium, and Sorghum. However, specific gRNAs are not available to target 41 (8.7%) and 40 (33.9%) of the NBS—LRR genes in Medicago and maize, respectively (Table 8). We reasoned that those NBS—LRR genes share a high level of sequence identity to other genomic sites because of their gene duplication and diversification history.

TABLE 8
Specific gRNA targetable NBS-LRR genes in eight plant species.

| | | No. of NBS-LRR | List of NBS-LRR |
|---|---|---|---|
| | No. of | genes | genes |
| | NBS-LRR | un-targetable | untargetable |
| Species | genes | by specific gRNAs | by specific gRNAs |
| Arabidopsis | 161 | 4 | AT1G58807, |
| thaliana | | | AT1G58848, |
| | | | AT1G59124, |
| | | | AT1G59218 |
| Medicago | 473 | 41 | Medtr1g024190, |
| truncatula | | | Medtr3g028040, |
| | | | Medtr3g044180, |
| | | | Medtr3g055010, |
| | | | Medtr3g055080, |
| | | | Medtr3g056360, |
| | | | Medtr3g056410, |
| | | | Medtr3g071070, |
| | | | Medtr4g019190, |
| | | | Medtr4g020730, |
| | | | Medtr4g020850, |
| | | | Medtr4g022960, |
| | | | Medtr4g043230, |
| | | | Medtr4g043500, |
| | | | Medtr4g043630, |
| | | | Medtr4g050790, |
| | | | Medtr4g050910, |
| | | | Medtr4g080320, |
| | | | Medtr4g080330, |
| | | | Medtr6g007830, |
| | | | Medtr6g072250, |
| | | | Medtr6g072290, |
| | | | Medtr6g072310, |
| | | | Medtr6g072320, |
| | | | Medtr6g073880, |
| | | | Medtr6g074030, |
| | | | Medtr6g074090, |
| | | | Medtr6g074170, |
| | | | Medtr6g074820, |
| | | | Medtr6g074840, |
| | | | Medtr6g075780, |
| | | | Medtr6g077590, |
| | | | Medtr6g079090, |
| | | | Medtr6g087260, |
| | | | Medtr6g088070, |
| | | | Medtr7g078300, |
| | | | Medtr8g038820, |
| | | | Medtr8g039870, |
| | | | Medtr8g043600, |
| | | | Medtr8g081370, |
| | | | Medtr8g087130, |
| Solanum | 161 | 1 | Solyc07g052800 |
| lycopersicum | | | |
| Glycine max | 502 | 11 | Glyma03g04040, |
| | | | Glyma03g06078, |
| | | | Glyma03g06271, |
| | | | Glyma03g06300, |
| | | | Glyma16g09963, |
| | | | Glyma18g09220, |
| | | | Glyma18g09824, |
| | | | Glyma18g09980, |
| | | | Glyma19g31662, |
| | | | Glyma19g31843, |
| | | | Glyma19g32090, |
| Brachypodium | 112 | 0 | |
| distachyon | | | |
| Oryza sativa | 395 | 2 | LOC_Os01g57310, |
| | | | LOC_Os12g29710 |
| Sorghum bicolor | 147 | 0 | |
| Zea mays | 118 | 40 | GRMZM2G002656, |
| | | | GRMZM2G003625, |
| | | | GRMZM2G003755, |
| | | | GRMZM2G005347, |
| | | | GRMZM2G005452, |
| | | | GRMZM2G006838, |
| | | | GRMZM2G016802, |
| | | | GRMZM2G017603, |
| | | | GRMZM2G028713, |
| | | | GRMZM2G045027, |
| | | | GRMZM2G047152, |
| | | | GRMZM2G050959, |
| | | | GRMZM2G051502, |
| | | | GRMZM2G065692, |
| | | | GRMZM2G074496, |
| | | | GRMZM2G076474, |
| | | | GRMZM2G077068, |
| | | | GRMZM2G078013, |
| | | | GRMZM2G079082, |
| | | | GRMZM2G094664, |
| | | | GRMZM2G116335, |
| | | | GRMZM2G150179, |
| | | | GRMZM2G167049, |
| | | | GRMZM2G173647, |
| | | | GRMZM2G176403, |
| | | | GRMZM2G322748, |
| | | | GRMZM2G327659, |
| | | | GRMZM2G379770, |
| | | | GRMZM2G396357, |
| | | | GRMZM2G397557, |
| | | | GRMZM2G401089, |
| | | | GRMZM2G443525, |
| | | | GRMZM2G444543, |
| | | | GRMZM2G452954, |
| | | | GRMZM2G454039, |
| | | | GRMZM2G461269, |
| | | | GRMZM2G549240, |
| | | | GRMZM5G837251, |
| | | | GRMZM5G880361, |
| | | | GRMZM5G898898 |

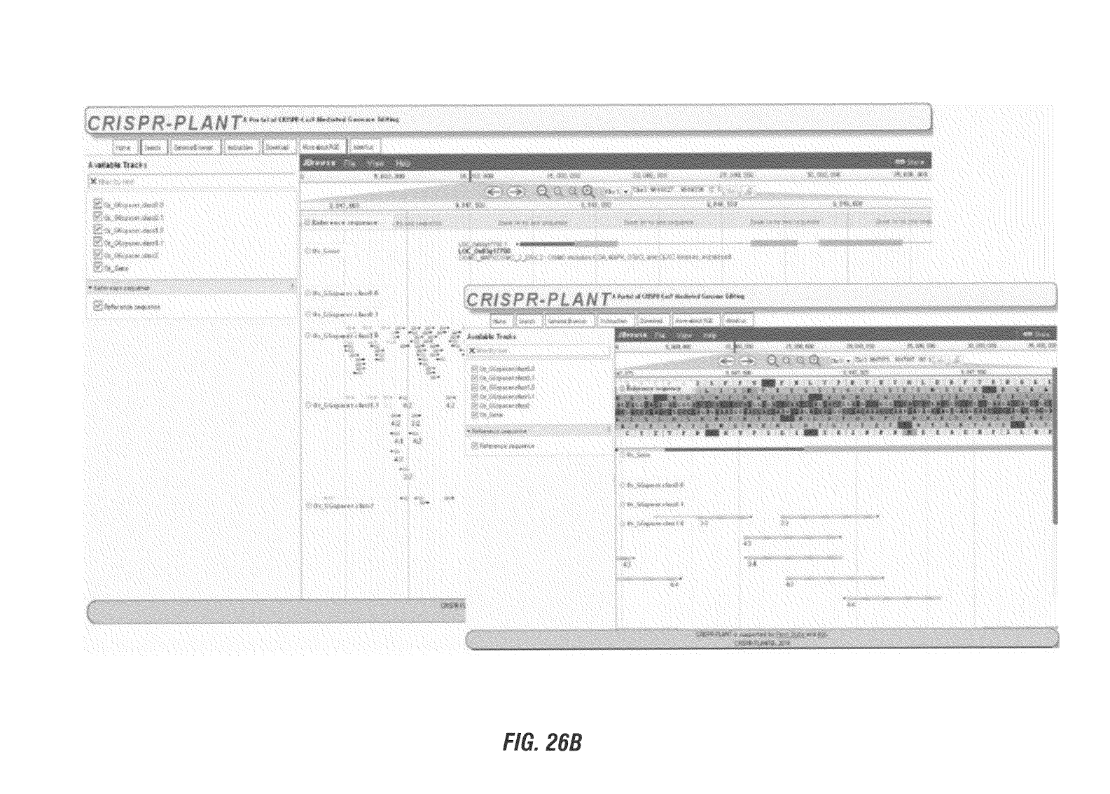

The genome-wide prediction of specific gRNA spacers suggests that the off-target effect is unlikely to constrain RGEb in most model plants and major crops, except maize. Besides maize, wheat and barley, which are important cereal crops with larger genome than maize, may also present a similar challenge for the CRISPR—Cas9-mediated RGE specificity. Considering the functional redundancy of some homologous genes with high sequence identity, specific gRNAs could be designed using spacer sequences other than Class0.0 or 1.0 to target duplicated genes without causing off-target effects to other transcripts. It was reported that Cas9 specificity was increased with a lower gRNA—Cas9 concentration (Hsu et al., 2013; Mali et al., 2013a; Pattanayak et al., 2013). Therefore, more gRNA spacer sequences, like some Class2 spacers, could be considered for specific RGE in practice. Alternative approaches such as the use of paired gRNAs and nickase mutation of Cas9 for reducing off-target risk (Mali et al., 2013a) or use of Cas9 orthologs recognizing different PAM may also help to increase specifically targetable sites, especially for maize. The Inventors have established the CRISPR-PLANT Database (www.genome.arizona.edu/crispr; FIG. 26) to enable the plant research community to access genome-wide predictions of specific gRNAs, and facilitate the application of CRISPR—Cas9-mediated genome editing in model plants and major agricultural crops.

### Methods

Analysis Pipeline

The bioinformatic analysis pipeline (FIG. 21B and FIG. 24) was modified from previously described analytical procedures (Xie and Yang, 2013). The pipeline used EMBOSS (Rice et al., 2000), USEARCH (Edgar, 2010), GASSST (Rizk and Lavenier, 2010), R/Bioconductor (Gentleman et al., 2004) and Bedtools (Quinlan and Hall, 2010) with customized PERL and R script to manipulate sequences and summarize results. The analysis was performed in the High Performance Computing Systems of the Pennsylvanian State University. The summary of analysis results is shown in Table 6.

Length of gRNA Spacer Sequence

Analysis was restricted to 20 nt long gRNA spacer sequences. The gRNA spacer sequence is identical to the sequence of the non-complementary DNA strand (protospacer) before the PAM of the targeting site (FIG. 21). Although longer gRNA spacer sequences could be used in genome editing, a recent report suggested that gRNAs with a longer spacer sequence were truncated in human cells and did not increase targeting specificity (Ran et al., 2013). Therefore, 20 nt long spacer sequences are appropriate for gRNA design and specificity assessment.

Extracting and Pre-Screening gRNA Spacer Sequence

For every genome, coordinates of PAMs (NGG or NAG) were identified in both strands of each chromosome using the pattern match program from EMBOSS. The 20 nt sequences immediately before the PAM, were then extracted from the same DNA strand of PAM, which resulted in two sequence sets: GG_spacer for NGGPAM and AG_spacer for NAG-PAM. All possible gRNA spacer sequences for Cas9 should be included in these two sequence sets, and the off-target potential of a spacer sequence could be estimated from its similarity to other GG_spacer and AG_spacer sequences. Because the affinity of Cas9 to NAG-PAM was much weaker than NGG-PAM (Hsu et al., 2013; Jiang et al., 2013a; Mali et al., 2013), the AG_spacer sequences were not considered for gRNA design in this study and was only used in GG_spacer off-target assessment. The following steps were taken to filter GG_spacer sequences to identify the candidates of specific gRNA spacer:

1) Hard masking was carried out to remove low complexity sequences. This step was carried out using USEARCH (Edgar, 2010) mask function and masked sequences were removed from candidates.

2) The 6-20 nt region of each spacer sequences was extracted and compared, and GG_spacers with identical sequence in 6-20 nt region were removed as multiple targeting spacers. Because the 15 by long gRNA-DNA pairing next to PAM is sufficient for Cas9 cleavage (Jinek et al., 2012), those spacers with identical 3′-end sequences of 15 nt long would recognize one another and should not be used to target unique site.

After these two steps, the remaining sequences from GG_spacer set were considered as candidates of specific gRNA spacer sequence.

Spacer Sequence Similarity Comparison

The off-target potential of selected GG_spacer candidates was evaluated by their similarity to all other spacer sequences. Total number of gaps (insertion/deletion) and nucleotides substitution in the sequences alignment were used for similarity measurement, which required pair-wised global alignment of each candidate with sequences from all GG_spacer and AG_spacer. Considering the computation cost of full implementation of pairwised global alignment is not feasible for millions of short sequences and is not necessary for gRNA spacer off-target evaluation, we set aligner tools to identify all alignments with less than 7 unmatched sites, either gaps or substitutions. The GASSST program, which is a sequence aligner based on Needle-Wunsch algorithm (Needleman and Wunsch, 1970) and allowed any number of gaps in alignment, was used for similarity comparison. GASSST was run with following settings: -r 0 -n 8 -p 70 -h 20. Because about 1% sequences failed to find the best hit in GASSST alignment, we also used the UBLAST to perform local alignment of candidates against all GG_spacers and AG_spacers. The UBLAST was run with following settings: -evalue 100 -self -strand plus. For big size genomes (>200 Mb), the UBLAST option -accel was set to 0.5 to reduce running time. It took 10 (Arabidopsis thaliana) to 100 (Zea mays) hours to complete the GASSST and UBLAST searching using twelve 64-bit 2.67 GHz CPUs. Alignment data from GASSST and UBLAST were combined and used for further analysis.

Classification of gRNA Spacer Sequences according to Targeting Specificity

Before processing alignment results, we removed the alignments in which both sequences were extracted from adjacent genomic sites containing consecutive PAM sites with less than 10 by spaced, because they are targeted adjacent position and should not be considered as “off-target” hits (sequence examples can be found in FIG. 24). For each alignment from GASSST or UBLAST, the total number of mismatches (including both gaps and substitutions) were extracted, and the minimal mismatches (minMM) from all GG_spacer alignments (minMM_GG) or all AG_spacer alignments (minMM_AG) for each candidate were calculated. Then candidate spacer sequences were classified according to their minMM value and mismatch position in alignments (FIG. 24).

1) Three classes of gRNA spacers were proposed based on their potential off-target effect on other NGG-PAM sites.
Class0 spacers were not aligned to other GG_spacer populations, and is expected to have no offtarget risk to other NGG-PAM site;
Class1 spacers have no fewer than 4 mismatches to other GG_spacer sequences (minMM_GG>=4), or have minimal 3 mismatches to other NGG-PAM sites (minMM_GG=3) but their 3′-end was not aligned with others in UBLAST alignments. They are also expected to cause no off-target risk to any other NGG-PAM site;
Class2 spacers are the remaining candidate sequences. They have a unique segment from 6-20 nt in their 3′-end (adjacent to PAM), but the mismatch number and position in GASSST/UBLAST alignments could not exclude them from the possibility of off-target risk to other NGG-PAM sites. Because class2 spacers aligned to off-targeted sites with mismatches, Cas9 expected to have less activity towards off-target sites than on-target sites.

2) A gRNA spacer candidate was considered to have no off-target risk to NAG-PAM site when it has not aligned to any AG_spacer or has no fewer than 3 mismatches when aligned with AG_spacer (minMM_AG>=3). Class0 and Class1 spacer sequences were further divided based on the following criteria:
Class0.0: Class0 spacers with no off-target risk to NAG-PAM site (minMM_AG>=3 OR not aligned with AG_spacer);
Class0.1: Class0 spacers with minMM_AG<3;
Class1.0: Class1 spacers with no off-target risk to NAG-PAM site (minMM_AG>=3 OR not aligned with AG_spacer);
Class1.1: Class1 spacers with minMM_AG<3.
   It is expected that gRNAs constructed from Class0.0 and Class1.0 spacer sequences should specifically guide Cas9 to unique genomic sites. Class0.1 and Class1.1 gRNAs have potential risk to off-target NAG-PAM sites. The number of spacer sequences in each processing step is shown in Table 15.

Mapping Cas9 Cleavage Sites in the Genome

The Cas9 cleavage position is located between the 4th and 3rd by before PAM (Jinek et al., 2012). A gRNA-Cas9 is designated to cut transcript unit/exon when the deduced Cas9 cleavage site is located in the transcript unit/exon or less than 3 bp away to the boundary of transcript unit/exon.

NBS-LRR Gene Family

To identify NBS-LRR genes in these eight plant species, the amino acid sequence of the conserved NBS domain was downloaded from the NIBLRRS Project website (http://niblrrs.ucdavis.edu/At_RGenes/HMM_Model/HMM_Model_NBS_Ath.html). This conserved sequence was used to search against the protein sequences of each species using BLASTP program. Homologous proteins with expect value less than 1.0×10-5 were considered as members of the NBS-LRR family.

CRISPR-PLANT Database

An online database of CRISPR-PLANT was established based on our analyzed data which could be accessed from: http://www.genome.arizona.edu/crispr. In CRISPR-PLANT, we provide gRNA spacer sequence information and analytical tools to help researchers to design and construct specific gRNAs for the CRISPR-Cas9 mediated plant genome editing (FIG. 26). Analysis results also can be viewed in the genome browser (FIG. 26) with the support of JBrowse (Skinner et al., 2009).

What is claimed is:

1. A method of altering expression of at least one gene product comprising introducing into a plant cell product an engineered, non-naturally occurring gene editing system comprising one or more vectors, said plant cell containing and expressing a DNA molecule having a target sequence and encoding the gene, said method comprising:
(a) a first regulatory element operable in a plant cell operably linked to at least one nucleotide sequence encoding a CRISPR-Cas system guide RNA (gRNA) that hybridizes with the target sequence, and
(b) a second regulatory element operable in a plant cell operably linked to a nucleotide sequence encoding a Type-II CRISPR-associated nuclease,
wherein components (a) and (b) are located on same or different vectors of the system, whereby the guide RNA targets the target sequence and the CRISPR-associated nuclease cleaves the DNA molecule, whereby expression of the at least one gene product is altered; and, wherein the CRISPR-associated nuclease and the guide RNA do not naturally occur together.

2. The method of claim 1 wherein said sequence encoding a gRNA and said sequence encoding a Type-II CRISPR-associated nuclease are operably linked to a terminator sequence functional in a plant cell.

3. The method of claim 1 wherein said type II CRISPR-associated nuclease is Cas9.

4. The method of claim 1 wherein said plant is Arabidopsis thaliana, Medicago truncatula, Solanum lycopersicum, Glycine max, Brachypodium distachyon, Oryza sativa, Sorghum bicolor, Zea mays, or Solanum tuberosum.

5. The method of claim 1 wherein said first regulatory element comprises a DNA-dependent RNA polymerase III (Pol III) promoter sequence.

6. The method of claim 5 wherein said Pol III promoter sequence is derived from a monocot plant.

7. The method of claim 6 wherein said Pol III promoter comprises a rice snoRNA U3 or U6 promoter nucleotide sequence.

8. The method of claim 6 wherein said Pol III promoter comprises a rice UBI10 promoter nucleotide sequence having at least 90% homology over its entire length to SEQ ID NO:1.

9. The method of claim 5 wherein said Pol III promoter sequence is derived from a dicot plant.

10. The method of claim 9 wherein said Pol III promoter sequence is a U3 promoter from Arabadopsis thaliana.

11. The method of claim 7 wherein said nucleic acid construct further comprises a multiple cloning site (MCS) located between the Pol III promoter and the gRNA sequence.

12. The method of claim 1 wherein said second regulator element comprises a DNA-dependent RNA polymerase II (Pol II).

13. The method of claim 1 wherein said nucleic acid construct further comprises a 15-30 by long DNA sequence inserted into the MCS site of the nucleic acid construct, wherein said 15-30 by long DNA sequence is complementary to the targeted genomic DNA sequence.

14. The method of claim 1 further comprising selecting said targeted genomic DNA sequence, wherein said selecting comprises identifying a protospacer-adjacent motif (PAM) in complementary strand of gene of interest.

15. The method of claim 10 further comprising engineering said gRNA to be complementary to the selected target, wherein the 5′-end of said engineered gRNA is adjacent to said PAM.

16. The method of claim 1 wherein said introducing results in transient expression of said sequences.

17. The method of claim 6 wherein said expression is in a plant cell protoplast.

18. The method of claim 1 wherein said introducing results in incorporation of said construct into the genome of said plant cell.

19. The method of claim 18 wherein said introduction comprises Agrobacterium-mediated transformation of said plant cell.

20. A modified plant cell produced by the method of claim 1.

21. A plant comprising the plant cell of claim 20.

22. Seed of the plant of claim 21.

23. The method of claim 1 wherein said alteration of expression of the at least one gene product confers one or more of the following traits: herbicide tolerance, drought tolerance, male sterility, insect resistance, abiotic stress tolerance, modified fatty acid metabolism, modified carbohydrate metabolism, modified seed yield, modified oil percent, modified protein percent, and resistance to bacterial disease, fungal disease or viral disease.

24. The method of claim 1 wherein components (a) and (b) are located on the same vector of the system, wherein said vector is at least 90% homologous over its entire length to one of pRGE3 (SEQ ID NO:2), pRGE6 (SEQ ID NO:4), pRGE31 (SEQ ID NO:6), pRGE32 (SEQ ID NO:8), pStGE3 (SEQ ID NO:10), pRGEB3 (SEQ ID NO:3), pRGEB6 (SEQ ID NO:5), pRGEB31 (SEQ ID NO:7), pRGEB32 (SEQ ID NO:9), or pStGEB3 (SEQ ID NO:11).

25. A nucleic acid construct for producing RNA-guided genome editing in plants, comprising:
(a) a first regulatory element operable in a plant cell operably linked to at least one nucleotide sequence encoding a CRISPR-Cas system guide RNA (gRNA) that hybridizes with the target sequence, and
(b) a second regulatory element operable in a plant cell operably linked to a nucleotide sequence encoding a Type-II CRISPR-associated nuclease,
wherein components (a) and (b) are located on same or different vectors of the system, whereby the guide RNA targets the target sequence and the CRISPR-associated nuclease cleaves the DNA molecule, whereby expression of the at least one gene product is altered; and, wherein the CRISPR-associated nuclease and the guide RNA do not naturally occur together.

26. The nucleic acid construct of claim 25 wherein said sequence encoding a gRNA and said sequence encoding a Type-II CRISPR-associated nuclease are operably linked to a terminator sequence functional in a plant cell.

27. The nucleic acid construct of claim 25 wherein said type II CRISPR-associated nuclease is Cas9.

28. The nucleic acid construct of claim 25 wherein said first regulatory element comprises a DNA-dependent RNA polymerase III (Pol III) promoter sequence.

29. The nucleic acid construct of claim 28 wherein said Pol III promoter sequence is derived from a monocot plant.

30. The nucleic acid construct of claim 29 wherein said Pol III promoter comprises a rice snoRNA U3 or U6 promoter nucleotide sequence.

31. The nucleic acid construct of claim 29 wherein said Pol III promoter comprises a rice UBI10 promoter nucleotide sequence having at least 80% homology over its entire length to SEQ ID NO:1.

32. The nucleic acid construct of claim 28 wherein said Pol III promoter sequence is derived from a dicot plant.

33. The nucleic acid construct of claim 31 wherein said Pol III promoter sequence is a U3 promoter from Arabadopsis thaliana.

34. The nucleic acid construct of claim 27 wherein said nucleic acid construct further comprises a multiple cloning site (MCS) located between the Pol III promoter and the gRNA sequence.

35. The nucleic acid construct of claim 25 wherein said second regulator element comprises a DNA-dependent RNA polymerase II (Pol II).

36. The nucleic acid construct of claim 25 wherein said nucleic acid construct further comprises a15-30 by long DNA sequence inserted into the MCS site of the nucleic acid construct, wherein said 15-30 by long DNA sequence is complementary to the targeted genomic DNA sequence.

37. The nucleic acid construct of claim 25 wherein components (a) and (b) are located on the same vector of the system, wherein said vector is at least 90% homologous over its entire length to one of pRGE3 (SEQ ID NO:2), pRGE6 (SEQ ID NO:4), pRGE31 (SEQ ID NO:6), pRGE32 (SEQ ID NO:8), pStGE3 (SEQ ID NO:10), pRGEB3 (SEQ ID NO:3), pRGEB6 (SEQ ID NO:5), pRGEB31 (SEQ ID NO:7), pRGEB32 (SEQ ID NO:9), or pStGEB3 (SEQ ID NO:11).


SEQUENCE LISTING (51 sequences)

SEQ ID NO: 1 - DNA, 1716 bp, Oryza sativa
acaaattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagat

SEQ ID NO: 2 - DNA, 9191 bp, Artificial Sequence
  Exemplary plamsid vector for transient transfection.
cttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggcactcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggttggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagcttt

SEQ ID NO: 3 - DNA, 15005 bp, Artificial Sequence
  Exemplary plasmid vector for stable transformation.
agcggataacaatttcacacaggaaacagctatgaccatgattacgccaagcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggttggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagctttcttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaagcactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtg

SEQ ID NO: 4 - DNA, 9552 bp, Artificial Sequence
  Exemplary plasmid vector for transient transformation.
cttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggcactcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctcattagcggtatgcatgttggtagaagtcggagatgtaaataattttcattatataaaaaaggtacttcgagaaaaataaatgcatacgaattaattctttttatgttttttaaaccaagtatatagaatttattgatggttaaaatttcaaaaatatgacgagagaaaggttaaacgtacggcatatacttctgaacagagagggaatatggggtttttgttgctcccaacaattcttaagcacgtaaaggaaaaaagcacattatccacattgtacttccagagatatgtacagcattacgtaggtacgttttctttttcttcccggagagatgatacaataatcatgtaaacccagaatttaaaaaatattctttactataaaaattttaattagggaacgtattattttttacatgacaccttttgagaaagagggacttgtaatatgggacaaatgaacaatttctaagaaatgggcatatgactctcagtacaatggaccaaattccctccagtcggcccagcaatacaaagggaaagaaatgagggggcccacaggccacggcccacttttctccgtggtggggagatccagctagaggtccggcccacaagtggcccttgccccgtgggacggtgggattgcagagcgcgtgggcggaaacaacagtttagtaccacctcgctcacgcaacgacgcgaccacttgcttataagctgctgcgctgaggctcaggttggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagcttt

SEQ ID NO: 5 - DNA, 15366 bp, Artificial Sequence
  Exemplary plasmid vector for stable transformation.
aaacagctatgaccatgattacgccaagcttctcattagcggtatgcatgttggtagaagtcggagatgtaaataattttcattatataaaaaaggtacttcgagaaaaataaatgcatacgaattaattctttttatgttttttaaaccaagtatatagaatttattgatggttaaaatttcaaaaatatgacgagagaaaggttaaacgtacggcatatacttctgaacagagagggaatatggggtttttgttgctcccaacaattcttaagcacgtaaaggaaaaaagcacattatccacattgtacttccagagatatgtacagcattacgtaggtacgttttctttttcttcccggagagatgatacaataatcatgtaaacccagaatttaaaaaatattctttactataaaaattttaattagggaacgtattattttttacatgacaccttttgagaaagagggacttgtaatatgggacaaatgaacaatttctaagaaatgggcatatgactctcagtacaatggaccaaattccctccagtcggcccagcaatacaaagggaaagaaatgagggggcccacaggccacggcccacttttctccgtggtggggagatccagctagaggtccggcccacaagtggcccttgccccgtgggacggtgggattgcagagcgcgtgggcggaaacaacagtttagtaccacctcgctcacgcaacgacgcgaccacttgcttataagctgctgcgctgaggctcaggttggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagctttcttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaacatctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacagga

SEQ ID NO: 6 - DNA, 9188 bp, Artificial Sequence
  Exemplary plasimd vector for trasnsient transformation
cttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggcactcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagcttt

SEQ ID NO: 7 - DNA, 15001 bp, Artificial Sequence
  Exemplary plasmid vector for stable transformation.
agcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccccagattagccttttcaatttcagaaagaatgctaacccacagatggttagagaggcttacgcagcagcactcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagcaggctccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagctttcttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaagcactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 8 - DNA, 10092 bp, Artificial Sequence
  Exemplary plasmid vector for transient transformation, incorporating novel OsUBI10 promoter.
cttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggcactcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccacaaattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatagttatcaacaagtttgtacaaaaaagcaggcttcgaaggagatagaaccaattctctaaggaaatacttaaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagcttt

SEQ ID NO: 9 - DNA, 15905 bp, Artificial Sequence
  Exemplary plasmid vector for stable transformation, incorporating novel OsUBI10 promoter.
cttgtacaaagtggttgataacagcgactacaaggatgacgatgacaaggcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaagcactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcatgcctgcaggtccacaaattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatagttatcaacaagtttgtacaaaaaagcaggcttcgaaggagatagaaccaattctctaaggaaatacttaaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcgcggccgcactcgagatatctagacccagcttt

SEQ ID NO: 10 - DNA, 8678 bp, Artificial Sequence
  Exemplary plasmid vector for transient transformation of dicots.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctctaagccttgtcatcgtcatccttgtagtcgctgttatcaaccactttgtacaagaaagctgggtctagatatctcgagtgcggccgcgaattcttactttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatggtggagcctgcttttttgtacaaacttgttgataactctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagtgctgctgcgtaagcctctctaaccatctgtgggttagcattctttctgaaattgaaaaggctaatctggggacctggtacccggggatcccagcctgtgatggataactgaatcaaacaaatggcgtctgggtttaagaagatctgttttggctatgttggacgaaacaagtgaacttttaggatcaacttcagtttatatatggagcttatatcgagcaataagataagtgggctttttatgtaatttaatgggctatcgtccatagattcactaatacccatgcccagtacccatgtatgcgtttcatataagctcctaatttctcccacatcgctcaaatctaaacaaatcttgttgtatatataacactgagggagcaacattggtcagagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagtcccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 14951 bp, Artificial Sequence
  Exemplary plasmid vector for stable transformation of dicots.
aattcgagctcggtacccgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctctaagccttgtcatcgtcatccttgtagtcgctgttatcaaccactttgtacaagaaagctgggtctagatatctcgagtgcggccgcgaattcttactttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatggtggagcctgcttttttgtacaaacttgttgataactctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagtgctgctgcgtaagcctctctaaccatctgtgggttagcattctttctgaaattgaaaaggctaatctggggacctggtacccggggatcccagcctgtgatggataactgaatcaaacaaatggcgtctgggtttaagaagatctgttttggctatgttggacgaaacaagtgaacttttaggatcaacttcagtttatatatggagcttatatcgagcaataagataagtgggctttttatgtaatttaatgggctatcgtccatagattcactaatacccatgcccagtacccatgtatgcgtttcatataagctcctaatttctcccacatcgctcaaatctaaacaaatcttgttgtatatataacactgagggagcaacattggtcagagaccgaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 12 - DNA, 44 bp, Oryza sativa
gaccatgattacgccaagcttctcattagcggtatgcatgttgg

SEQ ID NO: 13 - DNA, 36 bp, Oryza sativa
cgagacctcggtctccaacctgagcctcagcgcagc

SEQ ID NO: 14 - DNA, 41 bp, Oryza sativa
gaccatgattacgccaagcttaaggaatctttaaacatacg

SEQ ID NO: 15 - DNA, 37 bp, Oryza sativa
cgagacctcggtctccaacctgccacggatcatctgc

SEQ ID NO: 16 - DNA, 34 bp, Artificial Sequence
  Guide RNA scaffold DNA sequence amplification primer.
ggagaccgaggtctcggttttagagctagaaata

SEQ ID NO: 17 - DNA, 37 bp, Artificial Sequence
  Guide RNA scaffold DNA sequence amplification primer.
ggacctgcaggcatgcacgcgctaaaaacggactagc

SEQ ID NO: 18 - DNA, 38 bp, Artificial Sequence
  Primer for site-directed mutagenesis to remove Bsa I sites in vector.
gagaggcttacgcagcagcactcatcaagacgatctac

SEQ ID NO: 19 - DNA, 30 bp, Artificial Sequence
  Primer for site-directed mutagenesis to remove Bsa I sites in vector.
gccggtgagcgtggcactcgcggtatcatt

SEQ ID NO: 20 - DNA, 26 bp, Oryza sativa
ggttgtctacatcgccacggagctca

SEQ ID NO: 21 - DNA, 26 bp, Oryza sativa
aaactgagctccgtggcgatgtagac

SEQ ID NO: 22 - DNA, 24 bp, Oryza sativa
ggttgatcccgccgccgatccctc

SEQ ID NO: 23 - DNA, 24 bp, Oryza sativa
aaacgagggatcggcggcgggatc

SEQ ID NO: 24 - DNA, 26 bp, Oryza sativa
ggttgaagatgtcgtagagcaggtac

SEQ ID NO: 25 - DNA, 26 bp, Oryza sativa
aaacgtacctgctctacgacatcttc

SEQ ID NO: 26 - DNA, 21 bp, Oryza sativa
gccaccttccttcctcatccg

SEQ ID NO: 27 - DNA, 20 bp, Oryza sativa
gttgctcggcttcaggtcgc

SEQ ID NO: 28 - DNA, 22 bp, Oryza sativa
catcaggaaggttcgccagcac

SEQ ID NO: 29 - DNA, 24 bp, Oryza sativa
atcatatctggggtcggatagaac

SEQ ID NO: 30 - DNA, 20 bp, Oryza sativa
acagattgccccagcgagat

SEQ ID NO: 31 - DNA, 19 bp, Oryza sativa
tgtgagaaccccgcatcca

SEQ ID NO: 32 - DNA, 20 bp, Oryza sativa
ctatttccgctgcgaaccat

SEQ ID NO: 33 - DNA, 19 bp, Oryza sativa
agtgacggcgggtgctagg

SEQ ID NO: 34 - DNA, 22 bp, Oryza sativa
tggtcagtaatcagccagtttg

SEQ ID NO: 35 - DNA, 22 bp, Oryza sativa
caaatacttgacgaacagaggc

SEQ ID NO: 36 - DNA, 28 bp, Arabidopsis thaliana
taggatcccagcctgtgatggataactg

SEQ ID NO: 37 - DNA, 37 bp, Arabidopsis thaliana
cgagacctcggtctctgaccaatgttgctccctcagt

SEQ ID NO: 38 - DNA, 34 bp, Artificial Sequence
  Guide RNA scaffold DNA sequence amplification primer.
agagaccgaggtctcggttttagagctagaaata

SEQ ID NO: 39 - DNA, 27 bp, Artificial Sequence
  Guide RNA scaffold DNA sequence amplification primer.
tcaagcttcgcgctaaaaacggactag

SEQ ID NO: 40 - DNA, 28 bp, Artificial Sequence
  Primer for amplification of Cas9 gene fragment.
tcggtacccaggtccccagattagcctt

SEQ ID NO: 41 - DNA, 28 bp, Artificial Sequence
  Primer for amplification of Cas9 gene fragment.
tcggtaccgacgttgtaaaacgacggcc

SEQ ID NO: 42 - DNA, 24 bp, Solanum tuberosum
ggtcatatttcaatatggtgattt

SEQ ID NO: 43 - DNA, 24 bp, Solanum tuberosum
aaacaaatcaccatattgaaatat

SEQ ID NO: 44 - DNA, 24 bp, Solanum tuberosum
ggtcttccttctgtgttggtctcg

SEQ ID NO: 45 - DNA, 24 bp, Solanum tuberosum
aaaccgagaccaacacagaaggaa

SEQ ID NO: 46 - DNA, 20 bp, Solanum tuberosum
tcagttgaacctgcggaatt

SEQ ID NO: 47 - DNA, 20 bp, Solanum tuberosum
tcgatactcatggcaacatc

SEQ ID NO: 48 - DNA, 24 bp, Arabidopsis thaliana
ggttgcaaagtacctggctgatgc

SEQ ID NO: 49 - DNA, 24 bp, Arabidopsis thaliana
aaacgcatcagccaggtactttgc

SEQ ID NO: 50 - DNA, 26 bp, Arabidopsis thaliana
ggttatcaatgatcggttgcagtgga

SEQ ID NO: 51 - DNA, 26 bp, Arabidopsis thaliana
aaactccactgcaaccgatcattgat